(19) United States
(12) Patent Application Publication
(10) Pub. No.: US 2024/0208950 A1
(43) Pub. Date: Jun. 27, 2024

(54) COMPOSITIONS AND METHODS FOR TREATMENT OF CERVICAL AND OVARIAN CANCER

(71) Applicant: SOUTH DAKOTA BOARD OF REGENTS, Pierre, SD (US)

(72) Inventors: Fathi T. HALAWEISH, Brookings, SD (US); Subhash CHAUHAN, Brookings, SD (US); Mena JAGGI, Brookings, SD (US)

(21) Appl. No.: 18/292,049

(22) PCT Filed: Jul. 7, 2022
(86) PCT No.: PCT/US22/36324

§ 371 Date: Jan. 25, 2024

Related U.S. Application Data
(60) Provisional application No. 63/219,219, filed on Jul. 7, 2021.

(51) Int. Cl. C07D 405/12; A61K 31/4025; A61P 35/00

(52) U.S. Cl. CPC C07D 405/12; A61K 31/4025; A61P 35/00

(57) ABSTRACT

Provided are compositions and methods of using an anticancer agent for cancers, such as, ovarian and cervical cancer. The compositions and methods include ormeloxifene derivatives, a selective estrogen receptor modulator (SERM), for the treatment of ovarian and cervical cancers. The compositions and methods include various spectroscopy techniques and molecular modeling to produce derivative molecules comprising the ormeloxifene skeleton to enhance binding to key receptors implicated in cancer compared to ormeloxifene alone. The compositions and methods provide for reduced tumor volume and tumor weight compared to a placebo control.

### CROSS-REFERENCE TO RELATED APPLICATION(S)

This application claims priority to International PCT Application No. PCT/US2022/036324, filed on Jul. 7, 2022, which claims the benefit under 35 U.S.C. § 119(e) to U.S. Provisional Application 63/219,219, filed Jul. 7, 2021, and entitled Compositions and Methods for Treatment of Cervical and Ovarian Cancer, which is hereby incorporated herein by reference in its entirety for all purposes.

### FIELD OF THE INVENTION

Disclosed herein are methods and compositions for treating cancer.

### BACKGROUND OF THE INVENTION

Despite the recent advancement in diagnosis and improved strategies for treatment of cancer, it is still the leading cause of death for much of the world population. Incidence and death rate are increasing for several cancer types, which is costing billions of dollars every year. The total national expenditure for cancer care in the United States in 2010 alone was about 125 billion dollars and it is estimated to increase to about 173 billion dollars in 2020. Series of clinical studies and research on the fight against cancer have led to the discovery of many anticancer drugs and therapies. However, they are associated with drug resistant problems as well as serious side effects and high cost. Among these drugs is Tamoxifen, a molecular therapy targeting the estrogen receptor (ER) by a Selective Estrogen Receptor Modulator (SERM). The drug is effective in reducing the risk of breast cancer, but also found to have adverse side effects such as lower-limb lymphedema, increased risk of bone loss in premenopausal women, and has been linked to endometrial cancer in some women. This makes tamoxifen unsafe for the treatment of breast, ovarian, and other types of cancers. Therefore, there is an urgent need to design a new active molecular target anticancer agent for ovarian and cervical cancer to overcome the toxicity and cellular resistance problems.

### BRIEF SUMMARY OF THE INVENTION

In Example 1, an anticancer composition comprises a compound having the structure:

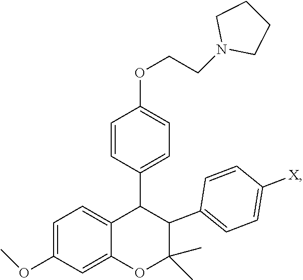

wherein x is an electron withdrawing group; or a salt, solvate, enantiomer, diastereoisomer, geometric isomer, or tautomer thereof.

Example 2 relates to the composition of Example 1, wherein the electron withdrawing group is selected from F, Cl, Br, and I.

Example 3 relates to the composition of Example 2, wherein the electron withdrawing group is Br.

Example 4 relates to the composition of any one of Examples 1-3, further comprising a pharmaceutically acceptable carrier.

Example 5 relates to the composition of any one of Examples 1-4, wherein the compound has greater binding affinity for at least one of EGFR, GSK3B, CDK2, STAT3 and/or mTOR, relative to the binding affinity of Ormeloxifene.

Example 6 relates to the composition of any one of Examples 1-5, wherein the composition is a Selective Estrogen Receptor Modulator (SERM).

Example 7 relates to the composition of Example 6, further comprising one or more anti-cancer agents.

In Example 8, a Selective Estrogen Receptor Modulator (SERM) comprises a compound having the structure:

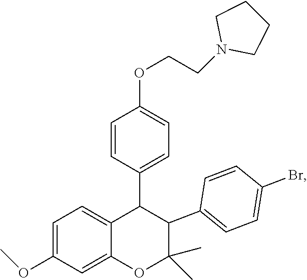 [SMILES: COc1ccc2c(c1)OC(C)(C)C(c1ccc(Br)cc1)C2c1ccc(OCCN2CCCC2)cc1]

and a pharmaceutically acceptable carrier thereof.

Example 9 relates to the composition of Example 8, wherein SERM binds to at least one of EGFR, GSK3B, CDK2, STAT3 and/or mTOR, with a binding affinity greater than that of Ormeloxifene.

In Example 10, a method for treating or preventing cancer in a subject in need thereof comprises administering to the subject an effective amount of a compound having the structure:

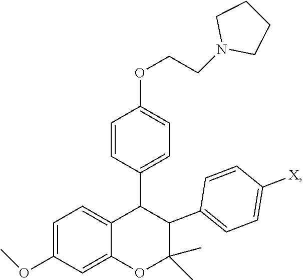

wherein x is an electron withdrawing group; or a salt, solvate, enantiomer, diastereoisomer, geometric isomer or tautomer thereof.

Example 11 relates to the method of Example 10, wherein the electron withdrawing group is selected from F, Cl, Br, and I.

Example 12 relates to the method of Example 11, wherein the electron withdrawing group is Br.

Example 13 relates to the method of any one of Examples 10-12, wherein the cancer is cervical cancer or ovarian cancer.

Example 14 relates to the method of any one of Examples 10-12, wherein the cancer is selected from melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-Hodgkin's lymphoma, hepatocellular carcinoma, bladder cancer, testicular cancer, oropharyngeal cancer, head and neck cancer, and lung cancer.

Example 15 relates to the method of any one of Examples 10-14, further comprising administering the composition in conjunction with at least one other treatment or therapy.

Example 16 relates to the method of Example 15, wherein the other treatment or therapy comprises co-administering at least one second anti-cancer agent.

Example 17 relates to the method of Example 16, wherein the co-administration of the composition and the at least one second anti-cancer agent provides a synergistic effect.

Example 18 relates to the method of any one of Examples 10-17, wherein the administration of the composition inhibits cancer cell migration in the subject.

Example 19 relates to the method of any one of Examples 10-18, wherein administration of the composition inhibits cancer cell invasion in the subject.

Example 20 relates to the method of any one of Examples 10-19, wherein the composition is administered by oral administration, intravenous administration, intradermal injection, intramuscular injection or subcutaneous injection.

### BRIEF DESCRIPTION OF THE FIGURES

FIG. 1 shows an example reaction scheme of the organic synthesis of the compounds of the present disclosure.

FIG. 2 shows the ability of ORM-series to inhibit the growth of A2780 cell lines.

FIG. 3A shows levels of protein of β-catenin in cyclohexamide-treated SiHa cells.

FIG. 3B shows levels of protein of β-catenin in ORM-Br and cyclohexamide-treated SiHa cells.

FIG. 4 shows the effect of ORM-Br on β-catenin degradation analyzed by pulse chase experiment.

FIG. 5A shows representative colony images of the effect of various concentrations of ORM-Br on CaSki cells.

FIG. 5B shows representative colony images of the effect of various concentrations of ORM-Br on SiHa cells.

FIG. 5C shows the quantification of colony formation in CaSki cells treated with various concentrations of ORM-Br.

FIG. 5D shows the quantification of colony formation in SiHa cells treated with various concentrations of ORM-Br.

FIG. 6 compares the cell survival of CaSki cells and SiHa cells when the cells are treated with various concentrations of ORM-Br.

FIG. 7A shows the effect of ORM-Br on the migration of CaSki cells.

FIG. 7B shows the effect of ORM-Br on the motility of CaSki cells.

FIG. 7C shows the effect of ORM-Br on the migration of SiHa cells.

FIG. 7D shows the effect of ORM-Br on the motility of SiHa cells.

FIG. 8A shows the effect of ORM-Br on invasion of CaSki.

FIG. 8B shows the effect of ORM-Br on invasion of SiHa cells.

FIG. 9A shows the effect of ORM-Br on β-catenin and beta-actin in a CaSki cytoplasmic extract.

FIG. 9B shows the effect of ORM-Br on β-catenin and Histone-H3 in a CaSki nuclear extract.

FIG. 9C shows the effect of ORM-Br on β-catenin and beta-actin in a SiHa cytoplasmic extract.

FIG. 9D shows the effect of ORM-Br on β-catenin and Histone-H3 in a SiHa cytoplasmic extract.

FIG. 9E shows the effect of ORM-Br on β-catenin localization in CaSki cells as illustrated by confocal microscopy.

FIG. 10 shows the effect of ORM-Br on EMT markers.

FIG. 11A shows exemplary images from molecular modeling studies showing ORM and ORM-Br bonding to different amino acids GSK3B binding pocket.

FIG. 11B shows exemplary images from molecular modeling studies showing ORM-Br forming hydrogen bonding with ASP:200:B in GSK3B binding pocket.

FIG. 12A shows exemplary images from molecular modeling studies showing ORM-Br in beta catenin binding pocket.

FIG. 12B shows exemplary images from molecular modeling studies showing Ormeloxifene and ORM-Br overlaying inside beta catenin binding pocket.

FIG. 13A shows bar graphs of the effect of Br-ORM on cell viability of (i) CaSki cells and (ii) SiHa cells.

FIG. 13B. shows the effect of Br-ORM on clonogenic potential of cervical cancer cells (i) CaSki and (ii) SiHa.

FIG. 13C. shows bar graphs indicating quantification of colony formation in (i) CaSki (ii) and SiHa cells of cervical cancer with respect to vehicle control.

FIG. 13D. shows the effect of Br-ORM on apoptosis in cervical cancer cells.

FIG. 13E. shows cervical cancer cells that were treated with the indicated concentrations of Br-ORM for 24 h; total cell lysates were prepared and subjected to Western blot analysis of apoptosis regulatory protein cleavage of PARP in both CaSki and SiHa cells.

FIG. 13F. shows the effect of Br-ORM on cell cycle progression in cervical cancer cells in both (i) CaSki and (ii) SiHa cells.

FIG. 13G shows bar graphs representing cell-cycle distribution in (i) CaSki and (ii) SiHa cells of cervical cancer.

FIG. 14A shows the effect of Br-ORM on cell migration of cervical cancer (CxCa) cells (i) CaSki and (ii) SiHa cells as determined by agarose bead.

FIG. 14B shows the effect of Br-ORM on cell migration of CxCa cells (i) CaSki and (ii) SiHa cells as determined by scratch wound healing

FIG. 14C shows the effect of Br-ORM on cell migration of CxCa cells as determined by Boyden chamber assay (i). The bar graph (ii) represents the quantification of migrated CaSki cells of control and Br-ORM treated groups.

FIG. 14D shows the effect of Br-ORM treatment on invasion of CaSki cells as determined by a commercially available kit (BD Biosciences) (i). The bar graph (ii) represents the quantification of invaded CaSki cells of control and Br-ORM treated groups.

FIG. 14E shows the effect of Br-ORM treatment on various EMT markers and MMPs in cervical cancer cells, (i) CaSki and (ii) SiHa cells.

FIG. 14F shows the effect on cervical cancer cells that were treated with Br-ORM for 18 h, where the RNA was isolated and subjected to qPCR for E-cadherin (i)-(ii), and N-cadherin (iii)-(iv).

FIG. 14G shows the effect of Br-ORM on E-cadherin expression in CxCa cells as determined by confocal microscopy in control and Br-ORM treated cells after 18 h.

FIG. 15A shows the effect of Br-ORM on β-catenin distribution in cytoplasm and nucleus of (i) and (iii) CaSki and (ii) and (iv) SiHa cells.

FIG. 15B shows the effect of Br-ORM on β-catenin localization in CaSki cells as determined by confocal microscopy for both (i)control and (ii) Br-ORM.

FIG. 15C shows representations of Br-ORM in-complexed with (i) β-catenin and (ii) residues participating in polar contacts and other interactions

FIG. 16A shows mRNA expression levels of HPV E6 and HPV E7 in (i) CaSki and (ii) SiHa cells.

FIG. 16B shows confocal images for nuclear staining showing HPV-E6, DAPI and the merged images of HPV-E6 and DAPI, using a Nikon confocal microscope.

FIG. 16C shows a representation of (i) Br-ORM in-complexed with E6 and (ii) residues participating in polar contacts and other interactions.

FIG. 16D shows (i) a schematic diagram of Br-ORM docking with HPV16 E7 showing residues involved in hydrogen-bonding and (ii) Van der Waals interactions and charge or polar interactions which are represented by varying shades.

FIG. 17A shows the effect of Br-ORM on (ii) CaSki cells derived xenograft tumors in (i) female athymic nude mice.

FIG. 17B is a line graph that indicates regression of CaSki cell-derived xenograft tumor volume in Br-ORM treated mice compared to control group.

FIG. 17C is a bar graph representing tumor weight of control and Br-ORM treated mice.

FIG. 17D shows the effect of Br-ORM on the expression of, (i) E-cadherin, (ii) β-catenin, (iii) Vimentin, (iv) Snail, (v) Slug, (vi) HPV E6, (vii) PCNA, and (viii) HPV E7 were further analyzed immunohistochemistry (IHC) in excised tumors of control and Br-ORM treated mice.

FIG. 17E shows the effect of Br-ORM on the expression of miR-200a in (i) control and (ii) Br-ORM treated mice excised tumors as determined by in situ hybridization.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

Ormeloxifene, is a selective estrogen receptor modulator (SERM), and has demonstrated excellent anti-cancer activity in different cancer cell lines including ovarian carcinoma, breast cancer, and head and neck squamous carcinoma with no life threatening side effects, however, there are concerns of cardiovascular side effects in some patients. Disclosed herein is a novel class of selective estrogen receptor modulators designed, synthesized and characterized by different spectroscopic techniques such as ¹H NMR, ¹³C NMR ¹³C-DEPT NMR, HSQC NMR and HRMS. Molecular modelling conducted against EGFR kinase domain revealed that, introduction of an electron withdrawing group at the para position of an aromatic ring at C-3 (ORM-Br) of an ormeloxifene skeleton increased binding affinity in receptors such as EGFR, GSK3B, CDK2, STAT3 and mTOR binding domain compared to ormeloxifene.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. As a further example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, “a subject in need thereof” means a human or non-human mammal that exhibits one or more symptoms or indications of cancer, and/or who has been diagnosed with cancer, including an ovarian cancer and/or cervical cancer, and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, and itching. The expression includes subjects with primary or established ovarian or cervical tumors,

In further embodiments, the expression “a subject in need thereof” includes patients with an ovarian or cervical cancer that is resistant to or refractory to or is inadequately controlled by prior therapy (e.g., treatment with a conventional anti-cancer agent). For example, the expression includes subjects who have been treated with chemotherapy, such as a platinum-based chemotherapeutic agent (e.g., cisplatin) or a taxol compound (e.g., docetaxel). The expression also includes subjects with an ovarian tumor for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with an ovarian tumor which has been treated but which has subsequently relapsed or metastasized. For example, patients with an ovarian tumor that may have received treatment with one or more anti-cancer agents leading to tumor regression; however, subsequently have relapsed with cancer resistant to the one or more anti-cancer agents (e.g., chemotherapy-resistant cancer) are treated with the compositions and methods disclosed herein.

As used herein, the term “synergistic effect” or grammatical variations thereof means and includes a cooperative action encountered in a combination of two or more active compounds in which the combined activity of the two or more active compounds exceeds the sum of the activity of each active compound alone.

The term “synergistically effective amount,” as used herein, means and includes an amount of two or more active compounds that provides a synergistic effect defined above.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present disclosure may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In preferred embodiments, the disclosed compositions are administered to the breast of a subject through intraductal injection. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The phrase “anti-cancer composition” can include compositions that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

### Pharmaceutical Compositions

The compounds discussed herein may be formulated using any convenient excipients, reagents and methods. In certain embodiments, there is provided a pharmaceutical composition comprising a compound having the structure:

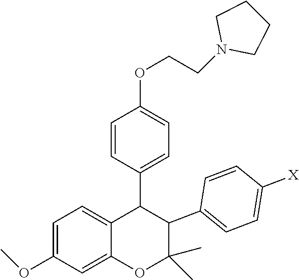

wherein X is an electron withdrawing group.

As used herein, an “electron withdrawing group” refers to an atom or group of covalently bonded atoms that draws electron density from neighboring atoms towards itself. In certain embodiments, electron withdrawing groups include, but are not limited to, halo (e.g. Br), halomethyl, polyhalomethyl, haloalkyl, polyhaloalkyl, aryl, haloaryl, polyhaloaryl, phenyl, benzyl, O-phenyl, cyano, ketone, aldehyde, amido, ester, hydroxy, methoxy, ether, alkene, alkyne, thio, thioether, thioester, nitro, nitroso, sulfonamido (—NH—SO₂-alkyl, —NH—SO₂-aryl, or —SO₂—NH—R where R can be H, alkyl, or aryl) and/or sulfonate (—O—SO₂—R, —SO₂—O—R, or —SO₂—R, wherein R can be alkyl or aryl but not H).

In certain aspects, the electron withdrawing group is selected from F, Cl, Br, and I. In further aspects, the electron withdrawing group is Br, also referred to herein as ORM-Br. In certain aspects, the foregoing compound has the structure:

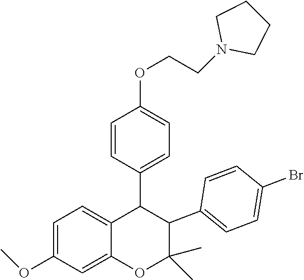 [SMILES: COc1ccc2c(c1)OC(C)(C)C(c1ccc(Br)cc1)C2c1ccc(OCCN2CCCC2)cc1]

In certain embodiments, the disclosed compound is a salt, solvate, enantiomer, diastereoisomer, geometric isomer or tautomer of the foregoing compounds.

In certain embodiments, the disclosed compositions bind one or more of the following targets (receptors) EGFR, GSK3B, CDK2, STAT3 and/or mTOR. In further embodiments, the disclosed compositions bind the forgoing targets with greater affinity than unmodified Ormeloxifene. In further embodiments, the disclosed compositions form hydrogen bonds within one or more binding pockets in the foregoing receptors.

In certain embodiments, compounds described herein are prepared as prodrugs. A “prodrug” refers to an agent that is converted into an active therapeutic compound in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure, are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining biodistribution or substrate receptor occupancy. Isotopically-labeled compounds are prepared by any suitable method or by processes using an appropriate isotopically-labeled reagent in place of the non-labeled reagent otherwise employed.

In yet further aspects, the composition further comprises a pharmaceutically acceptable excipient. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally, the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

The instant disclosure includes methods for treatment and/or prevention of cancer. As provided herein, a method for treating or preventing cancer includes administration of an effective amount of the disclosed compositions to a subject in need thereof.

According to certain embodiments, the subject in need thereof has been diagnosed with cancer. In exemplary embodiments, the subject has been diagnosed with cervical cancer. In further exemplary embodiments, the subject has been diagnosed with ovarian cancer.

According to further embodiments, the subject has been diagnosed with a cancer that includes, but is not limited to, squamous cell cancer (e.g., epithelial squamous cell cancer), melanoma, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, gastrointestinal stromal tumors, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, testicular cancer, hepatic carcinoma, anal carcinoma, penile carcinoma, mouth and throat cancer as well as head and neck cancer. In some embodiments the cancer is a carcinoma or sarcoma. In various embodiments, the cancer is a solid tumor, as these produce the most strongly acidic tumor microenvironment. In various embodiments, solid tumors can be defined to include certain circumstances of otherwise non-solid cancer cell masses, such as lymphoma building up as quasi-solid masses in lymph nodes and similar collection areas in the body.

The dosage administered will be dependent upon the age, health, and weight of the recipient, kind of concurrent treatment, if any, frequency of treatment, and the nature of the effect desired.

According to certain embodiments, the composition is administered in a prophylactically effective amount. According to exemplary aspects of these embodiments, the composition is administered to subjects at high risk of developing cervical and/or ovarian cancer.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a cancer in a patient.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, an inhalant, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

In some embodiments, the subject compound and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a chemotherapeutic agent.

In another aspect, a pharmaceutical composition is provided, comprising, or consisting essentially of, a compound disclosed herein, or a pharmaceutically acceptable salt, isomer, tautomer or prodrug thereof, and further comprising one or more additional active agents of interest. Any convenient active agents can be utilized in the subject methods in conjunction with the subject compounds. In some instances, the additional agent is a chemotherapeutic agent. The subject compound and chemotherapeutic agent, as well as additional therapeutic agents as described herein for combination therapies, can be administered orally, subcutaneously, intramuscularly, intranasally, parenterally, or other route. The subject compound and second active agent (if present) may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In certain cases, the therapeutic agents can be administered intranasally. In some cases, the therapeutic agents can be administered intratumorally.

In some embodiments, the subject compound and a chemotherapeutic agent are administered to individuals in a formulation (e.g., in the same or in separate formulations) with a pharmaceutically acceptable excipient(s). The chemotherapeutic agents include, but are not limited to alkylating agents, nitrosoureas, antimetabolites, antitumor antibiotics, plant (vinca) alkaloids, and steroid hormones. Peptidic compounds can also be used. Suitable cancer chemotherapeutic agents include taxane and active analogs and derivatives thereof, dolastatin and active analogs and derivatives thereof, and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD)).

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also suitable carriers.

Any drug delivery device or system that provides for the dosing regimen of the instant disclosure can be used. A wide variety of delivery devices and systems are known to those skilled in the art.

According to certain embodiments, the disclosed method further comprises administering the composition in conjunction with at least one other treatment or therapy. In certain aspects, the at least one other treatment or therapy comprises co-administering an anti-neoplastic agent. In certain aspects, the other treatment or therapy is chemotherapy.

According to certain further embodiments, the method further comprises diagnosing the subject with cancer. In further aspects, the subject is diagnosed with cancer prior to administration of the composition. According to still further aspects, the method further comprises evaluating the efficacy of the composition. In yet further aspects, evaluating the efficacy of the composition comprises measuring tumor size prior to administering the composition and measuring tumor size after administering the composition. In even further aspects, evaluating the efficacy of the composition occurs at regular intervals. According to certain aspects, the disclosed method further comprises optionally adjusting at least one aspect of the method. In yet further aspects, adjusting at least one aspect of the method comprises changing the dose of the composition, the frequency of administration of the composition, or the route of administration of the composition.

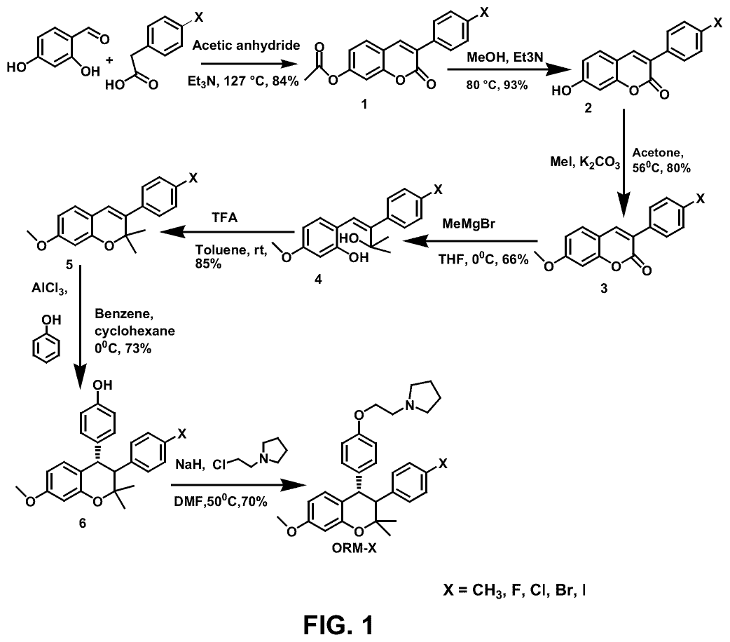

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described in Scheme 1 shown in FIG. 1.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

### Example 1

In one example, methyl, hydroxyl, amine and halogenated ormeloxifene were evaluated for biological activity. Table 1 shows a first series of ormeloxifene analogs.

TABLE 1

| | (X) = OH, NH₂, |
|---|---|
| Compound | Halogen, CH₃ |
| ORM-F | F |
| ORM-Cl | Cl |
| ORM-Br | Br |
| ORM-I | I |
| ORM-CH₃ | CH₃ |
| ORM-OH | OH |
| ORM-NH₂ | NH₂ |

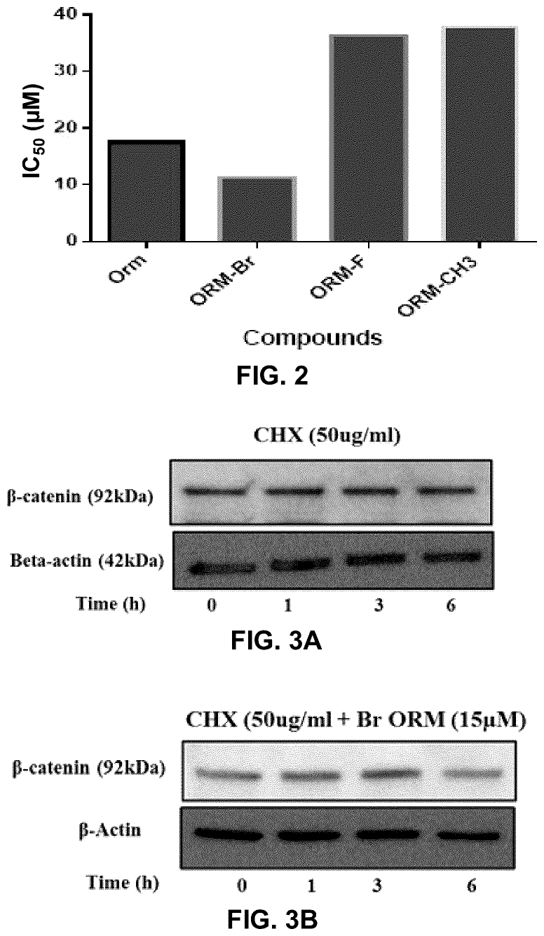

A biological assay was performed to study the potency of ORM-analogs by observing their pharmaceutical effects on living tissue (in vitro) and the effects of these analogs of unknown potency were compared to the effects of the standard (ormeloxifene). An MTT cell viable assay was first conducted to measure the cytotoxicity and anti-proliferation activity of synthesized analogs (Table 2 and FIG. 2).

TABLE 2

| Compound | IC₅₀ (μM) |
|---|---|
| Ormeloxifene | 17.5 μM |
| ORM-Br | 11.2 μM |
| ORM-F | 36.3 μM |
| ORM-Cl | NA |
| ORM-CH₃ | 37.9 μM |
| ORM-I | NA |
| ORM-OH | NA |

Among all the analogs, ORM-Br showed stronger cytotoxicity on ovarian cancer cell line A2780 with an IC₅₀ value of 11.2 μM relative to ormeloxifene which has an IC₅₀ of 17.5 μM (Table 2 and FIG. 2). However, compounds such as ORM-F and ORM-CH₃ showed a loss in cytotoxicity on ovarian cancer cell lines with IC₅₀ values of 36.3 μM and 37.9 μM respectively as compared to ormeloxifene. Compounds such as ORM-Cl, ORM-I and ORM-OH completely lost their cytotoxicity on ovarian cancer cell lines. These results demonstrated that, presence of bromine atom at the para position of an aromatic at C-3 improves the overall cytotoxicity activity of the compound on the ovarian cancer cell line.

To understand anti-proliferation mechanism of ORM-Br, a further biological assay was conducted using cervical cancer cell lines CaSki and SiHa. A western blot analysis was conducted to investigate the effect of ormeloxifene analogs on protein levels of an ovarian cancer cell line. Ovarian cancer cells (70-80% confluent) were treated with a 10-20 mmol/L concentration of ormeloxifene analogs for 72 hours. Control cells were treated with 0.1% DMSO. Total lysates were prepared via known techniques.

A western blot analysis was conducted to determine the effect of ORM-Br on β-catenin degradation. After using a translational inhibitor (cyclohexamide) a significant decrease in protein levels of β-catenin in CaSki and SiHa cells relative to cyclohexamide treatment was shown (FIGS. 3A and 3B). As shown in FIGS. 3A and 3B, SiHa cells were treated with cyclohexamide (CHX: 50 mg) alone or in combination with ORM-Br (15 mmol/L) at indicated time points. Protein lysates were prepared and subjected to western blot analysis to ascertain the levels of protein of β-catenin in cyclohexamide-treated cells (FIG. 3A) and in ORM-Br and cyclohexamide-treated cells (FIG. 3B).

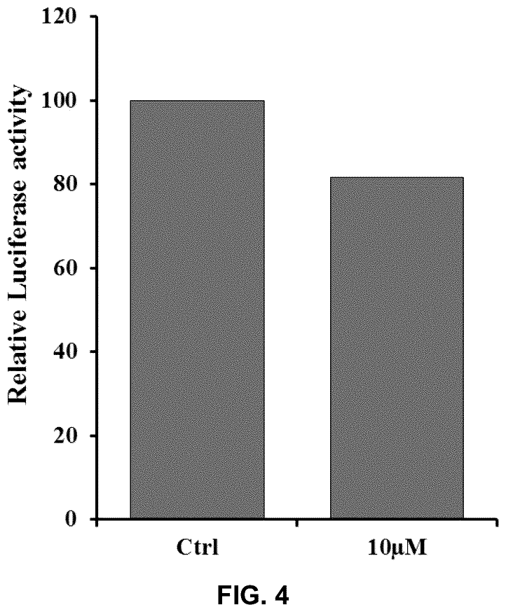

FIG. 4 shows the effect of ORM-Br on β-catenin degradation analyzed by a pulse chase experiment.

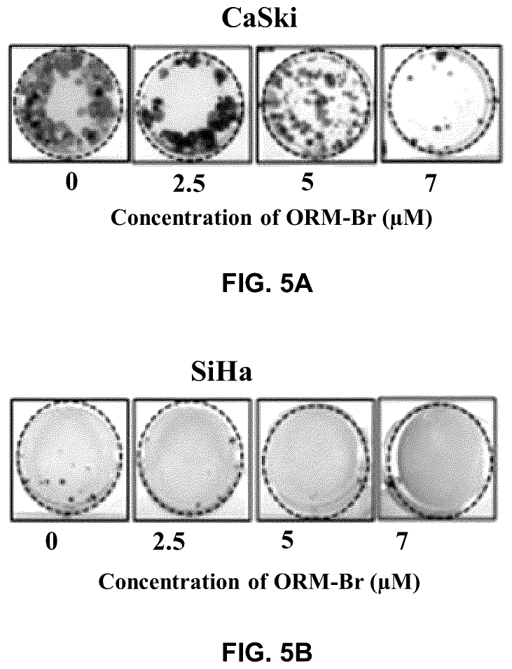

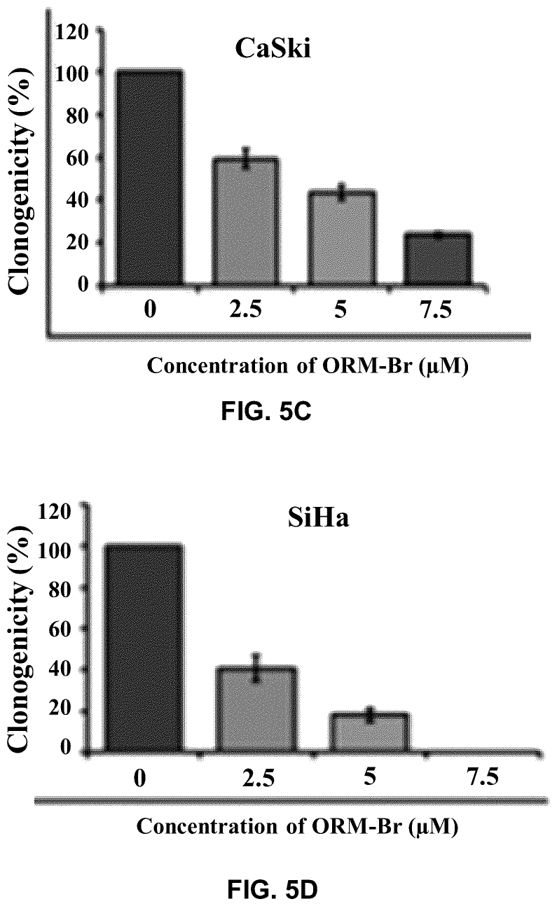

Further, as shown for example in FIGS. 5A-5D, ORM-Br suppresses the clonogenic potential of CaSki and SiHa cell lines. FIGS. 5A and 5B show representative colony images of CaSki cells (FIG. 5A) and SiHa cells (FIG. 5B) treated with various concentrations of ORM-Br ranging from 0 to 7 μM. FIGS. 5C and 5D represent the quantification of colony formation in CaSki cells (FIG. 5C) and SiHa cells (FIG. 5D) treated with various concentrations of ORM-Br ranging from 0 to 7.5 μM.

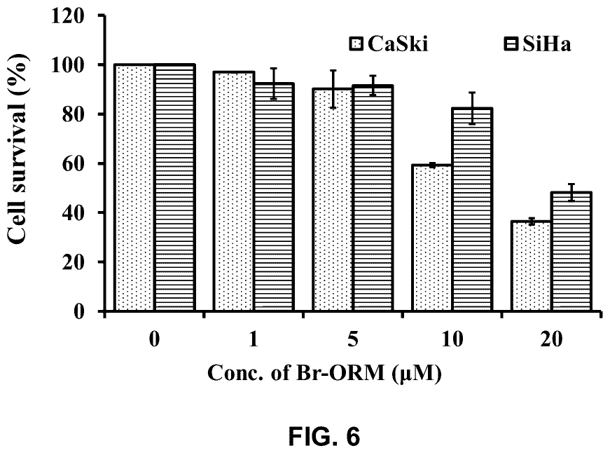

FIG. 6 compares the cell survival of CaSki cells and SiHa cells when the cells are treated with various concentrations of ORM-Br. As can be seen, ORM-Br inhibits the growth of cervical cancer cells—both CaSki and SiHa cells

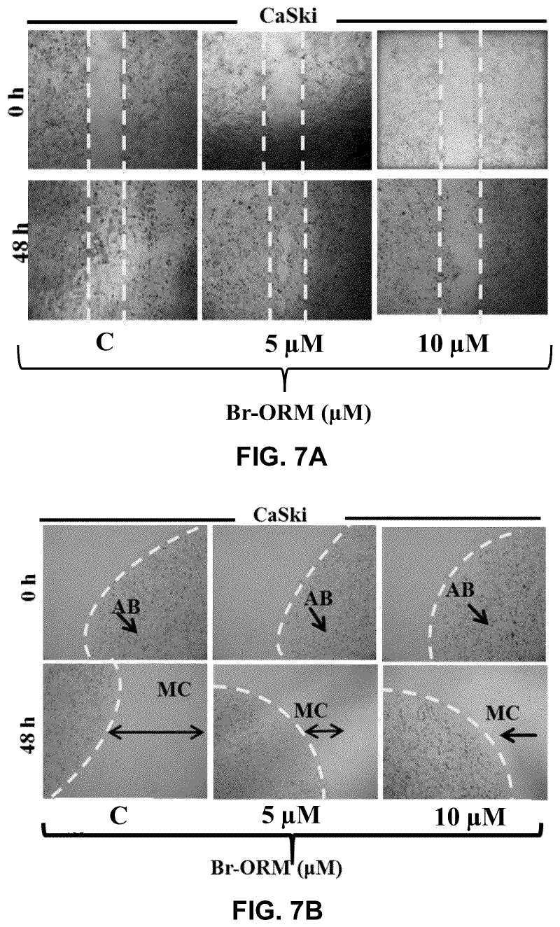

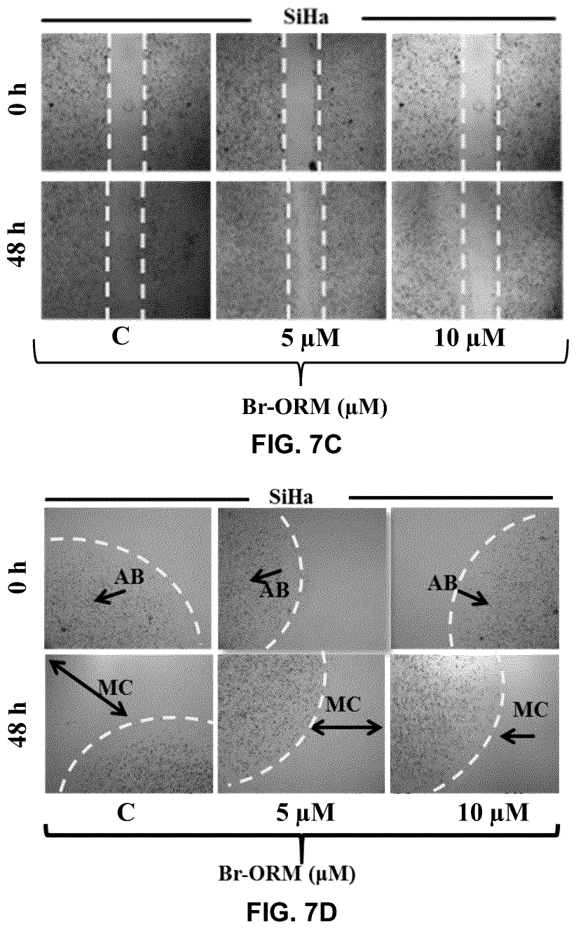

FIGS. 7A-7D shows the inhibition of the migration of cervical cancer cells treated with different concentrations of ORM-Br, as predicted by agarose bead assay. In FIGS. 7A and 7C, ORM-Br inhibits the migration of cervical cancer in CaSki cells (FIG. 7A) and SiHa cells (FIG. 7C). FIGS. 7B and 7D show the effect of ORM-Br on motility potential of CaSki cells (FIG. 7B) and SiHa cells (FIG. 7D) as predicted by agarose bead assay. The images shown in FIGS. 7A-7D represent the migratory cells (MC) in control and ORM-Br treated groups at 0 and 48 hours. In FIGS. 7B and 7D, AB means agarose beads. In FIGS. 7A-7D the images magnification were at 4.

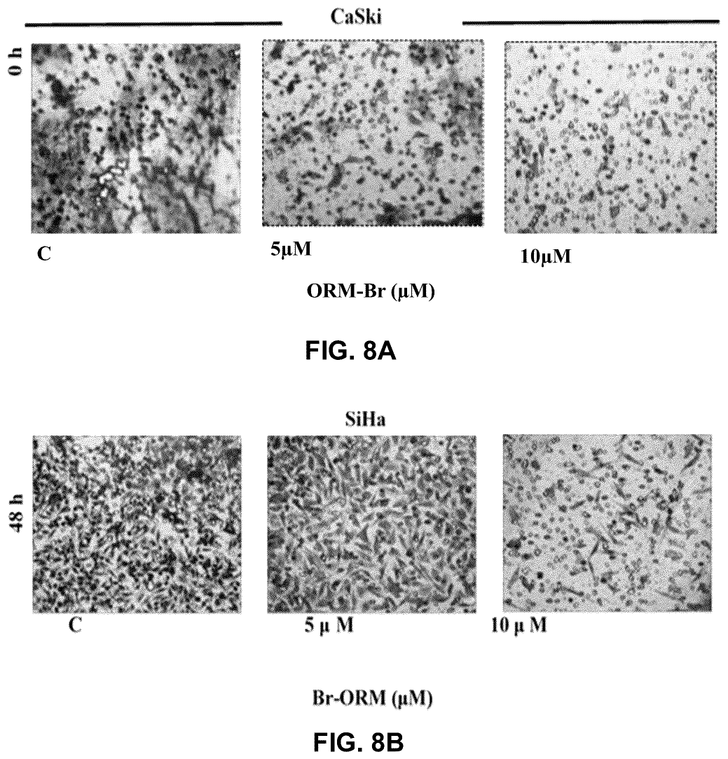

FIGS. 8A and 8B show the effect of ORM-Br on invasion of CaSki (FIG. 8A) and SiHa (FIG. 8B) cell lines in Boyden chamber and xCELLigence assay. The images represent the invaded cells of control and ORM-Br-treated CaSki and SiHa cells as determined by Boyden Chamber kit. The magnification was at 20 for the images of FIGS. 8A and 8B.

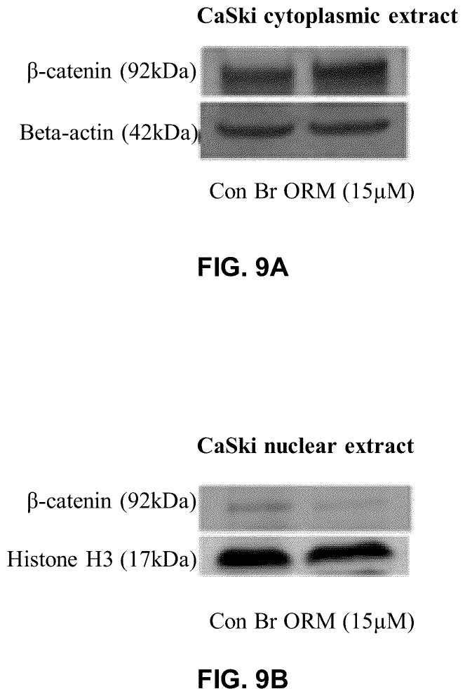

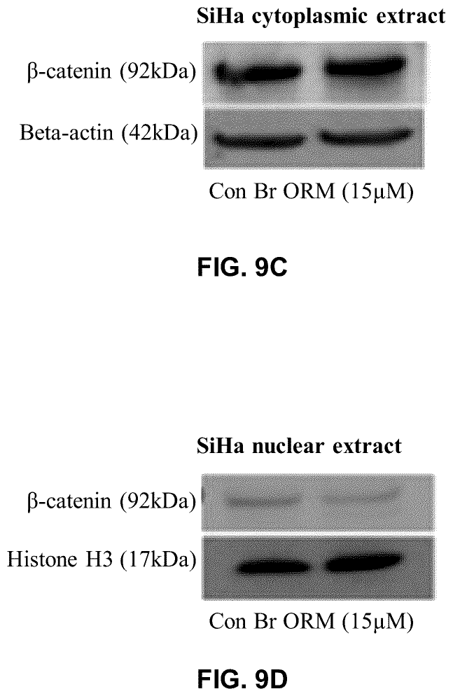

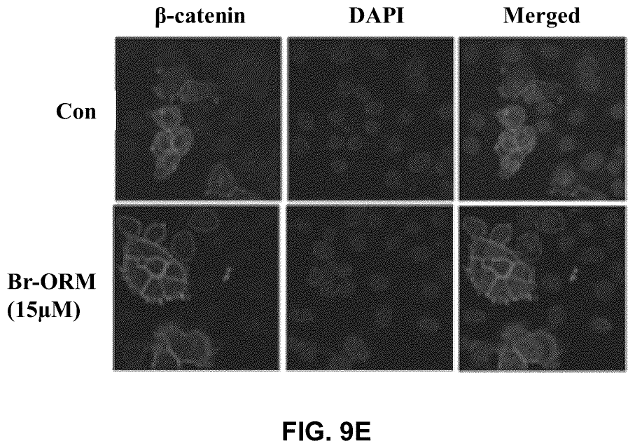

The Boyden chamber and xCELLigence assay revealed that ORM-Br inhibits the invasion of cervical cancer cells (FIGS. 9A-9E). In this example, there was a decrease in expression of nuclear β-catenin in the cytoplasm of cervical cancer cell lines when subjected to western blot analysis to detect the protein levels of β-catenin (FIGS. 9A and 9C).

In the Example shown at FIGS. 9A-9E, cells were subjected to treatment with 15 μM concentration of ORM-Br for 24 hours, nuclear extracts were prepared and western blot analysis were used to detect the protein levels of β-catenin. The results revealed a decrease in expression of nuclear β-catenin and increased expression of β-catenin in the cytoplasm of CaSki cells (FIG. 9A) and SiHa cells (FIG. 9C). Western blots were reprobed with β-actin and Histone H3 antibodies as an internal control. Effect of ORM-Br on β-catenin localization in CaSki cells as illustrated by confocal microscopy is shown in FIG. 9E.

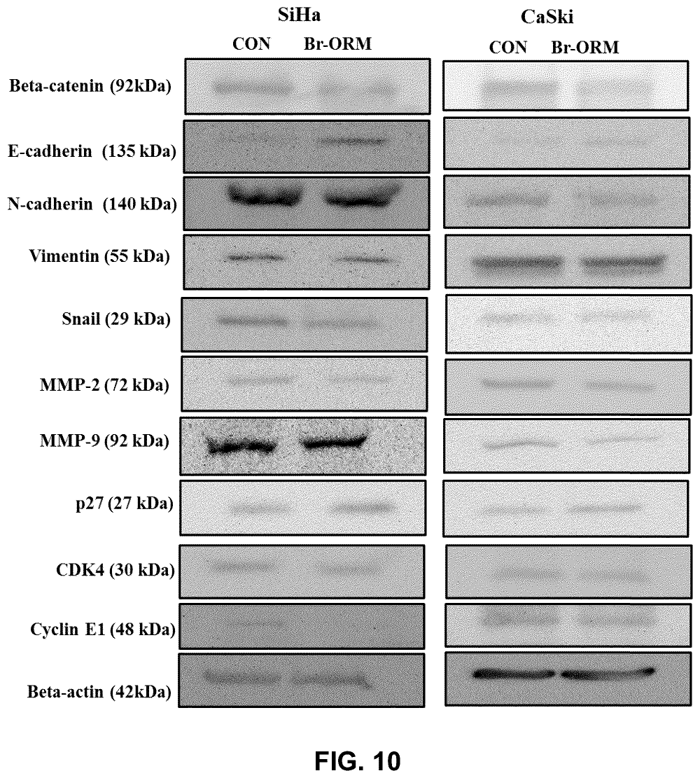

In a further example, cell lysates and western blot analysis was conducted for EMT markers and MMPs. The analysis showed that of ORM-Br inhibits the EMT associated markers in both CaSki and SiHa cell lines (FIG. 10). In this example, about 70% of confluent SiHa and CaSki were treated with ORM-Br (10-20 mmol/L) for 24 hours. Cell lysates were prepared, and western blot analysis were conducted for EMT markers and MMPs analysis. The effect of ORM-Br on protein levels of cell-cycle regulatory proteins (cyclin E1 and p27) in both CaSki and SiHa cell lines are shown.

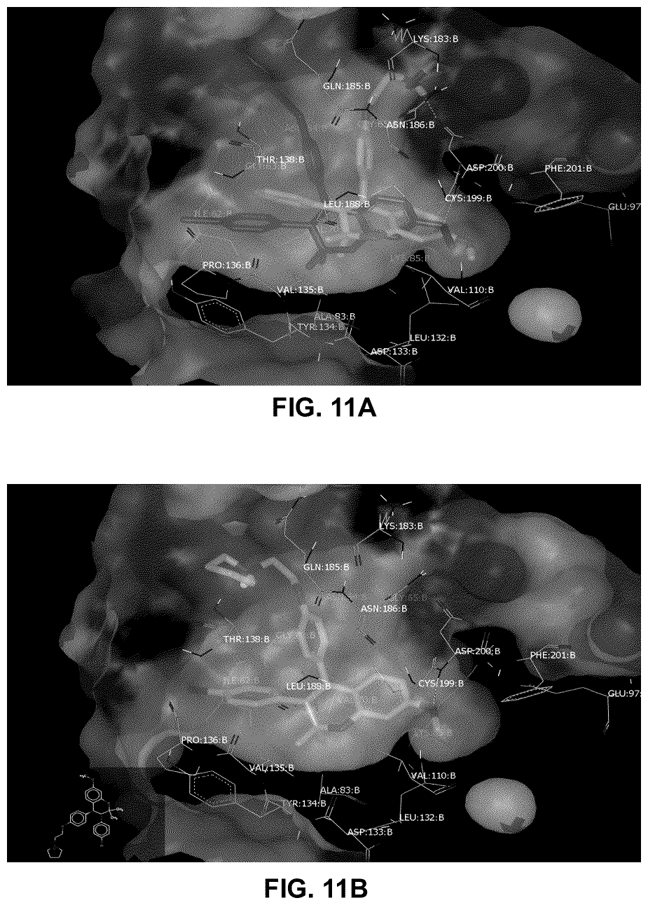

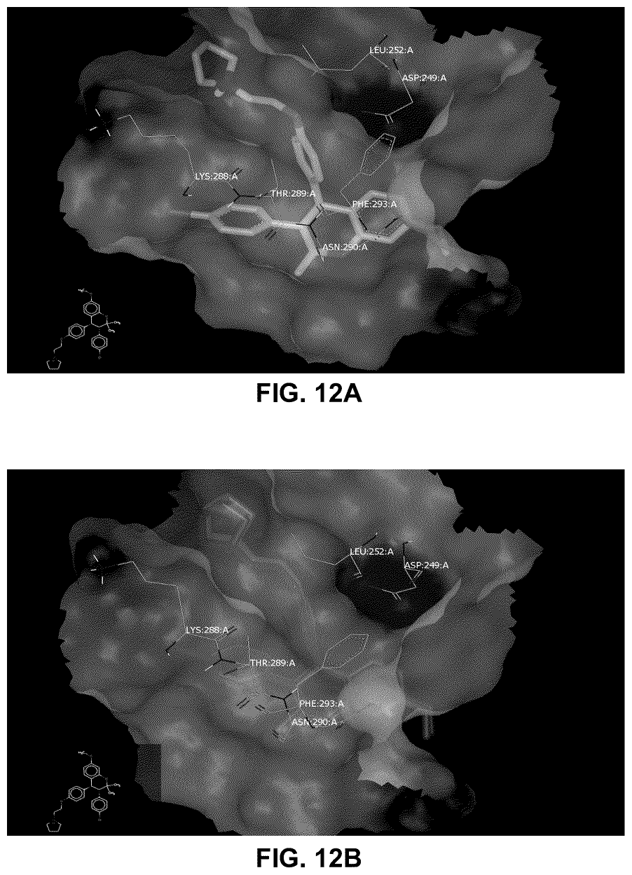

Molecular modeling studies were conducted to assess the projected interactions between the disclosed compounds and protein kinase targets. Molecular docking results revealed that introduction of electron donating (particularly methyl, amine and hydroxyl) groups at the aromatic ring on C-3, as in ORM-CH₃, ORM-OH, ORM-NH₂, increased the binding affinity two fold in the crystal structure of EGFR compared to ormeloxifene. FIG. 11A shows exemplary images from molecular modeling studies showing ORM and ORM-Br bonding to different amino acids GSK3B binding pocket. FIG. 11B shows ORM-Br forming hydrogen bonding with ASP: 200:B in GSK3B binding pocket. FIG. 12A shows ORM-Br in beta catenin binding pocket. FIG. 12B shows Ormeloxifene and ORM-Br overlaying inside beta catenin binding pocket.

As a result, analogs of ormeloxifene were designed using a structural based drug design (SBDD). Various structural modifications on ormeloxifene were made based on the most pharmacophores for its biological activity. Docking studies revealed that, installation of halogens on the ormeloxifene scaffold improved both binding affinity in GSK3B, EGFR and β-catenin binding pockets and, this revelation was also confirmed by biological evaluation. The IC₅₀ of ORM-Br (11.2 μM) was better than all other analogs including ormeloxifene in MTT analysis. Further biological studies confirmed that ORM-Br inhibits the localization and invasion of 3-catenin in cervical cancer cell lines.

### Example 2

### Materials and Methods

### Cell Lines and Growth Condition

HPV-16 positive human cervical cancer cells (CaSki and SiHa) were purchased from American Type Cell Culture (Manassas, VA) and cultured in RPMI-1640 and DMEM medium (HyClone Laboratories, INC., Logan, UT) supplemented with 10% heat-inactive FBS (Atlanta Biologicals, Norcross, GA), 1% penicillin, and 1% streptomycin (Gibco BRL, Grand Island, NY, USA) respectively. Cells were grown in a humidified atmosphere containing 5% CO₂ at 37° C.

### Chemicals and Antibodies

Specific monoclonal and polyclonal antibodies of β-actin (cat. no. 3700), Histone H3 (cat. no. 4499), GAPDH (cat. no. 5174), N-cadherin (cat. no. 4061), Slug (cat. no. 9585), Snail (cat. no. 3879), Vimentin (cat. no. 5741), PARP (cat. no. 9532S), MMP2 (cat. no. 4022), and MMP9 (cat. no. 13667) were obtained from Cell Signaling Technology Inc. β-catenin (cat. no. SC-7199) and E-cadherin (cat. no. SC-7870) antibodies were obtained from Santa Cruz Biotechnology. Horseradish peroxidase (HRP)-conjugated anti-mouse (cat. no. 4021) and anti-rabbit (cat. no. 4011) antibodies were acquired from Promega (Madison, WI, USA). Anti-mouse Cy3 secondary antibody was purchased from Thermo Fisher Scientific. HPV E6 (cat. no. ab70) specific antibody was procured from Abcam Cambridge, MA.

### Cell Proliferation Assay

The effect of Br-ORM on CaSki and SiHa cells proliferation was performed using the MTS assay. Briefly, cells were seeded at a density of 5×10³ cells per well in 96 well plate and allowed to stand overnight at 37° C. and 5% CO₂ incubator. Next day, cells were treated with different concentrations of Br-ORM (1, 5, 10 and 20 μM). DMSO was used as a vehicle for the treatment of control cells. Twenty four hours post-treatment, 20 μL of MTS reagent (5 mg/mL) was added in each well and further incubated the plate for 2 hours in a CO₂ incubator. Absorbance was taken after 2 hours at 570 nm (SpectraMax M2 spectrophotometer, Molecular Devices, Sunnyvale, CA, USA). The experiment was performed in triplicates. Results were represented as percent viability with respect to the control group.

### Colony Formation Assay

To determine the effect of Br-ORM on clonogenic potential of CaSki and SiHa cells, a colony formation assay was performed. In brief, 500 cells were seeded per well in a 6-well plate and allowed to stand for the next three days. The cells were treated with Br-ORM with different concentrations (2.5, 5, and 7.5 μM) for seven days and then followed by normal growth media. DMSO was used as a vehicle for the treatment of control cells. Colonies were fixed in methanol, stained with haematoxylin, and counted using UVP 810 software.

### Apoptosis Analysis

The apoptosis inducing effect of Br-ORM on cervical cells was analyzed by Annexin V-FLUOS staining kit (Roche Diagnostic Corp. Indianapolis, IN, USA). The procedure was followed as described in the vendor's protocol. Briefly, 60% confluent cervical cancer cells (CaSki and SiHa) were treated with Br-ORM (10, 15, and 20 μM) and kept in a CO₂ incubator at 37° C. for 24 hours. Control group cells were treated with DMSO as the vehicle. Cells were washed with PBS (1×) and kept in Annexin-V solution for 20 minutes. Images were captured in bright and green field by fluorescent microscope.

### Cell Cycle Analysis

The effect of Br-ORM on cell cycle analysis was performed by flow cytometry as has described in the art. In brief, approximately 70% confluent cervical cancer cells were treated with Br-ORM (10 and 20 μM) for 24 hours. The cells were trypsinized and washed twice with ice-cold PBS (1×). The cell pellets were resuspended in 50 μL of ice cold PBS (1×) and 450 μL of cold methanol. The cells were washed twice with ice cold PBS (1×), suspended in 500 μL PBS, and incubated with 500 μL RNase (20 μg/mL final concentration) at 37° C. for 1 hour. The cells were chilled over ice for 10 minutes and stained with propidium iodide (50 μg/mL final concentration) for 1 hour and analyzed by flow cytometry (BD Accuri C6; Becton Dickinson, Mountain View, CA, USA). Data was analyzed by using Modfit software.

### Cell Migration Assay

Cell motility was performed by in vitro scratch wound assay. Briefly, cells were seeded in a 12-wells plate and after 80-90% confluency a standardized wound was made using a 200 μL micropipette tip. Cells were then treated with Br-ORM (10 μM) and photographed by phase contrast microscopy.

### Agarose Bead Assay

Cells migration was performed by agarose bead assay as has been described in the art. Briefly, cells were mixed into a low melting point agarose solution and drops of suspension were placed onto plates. Cells were treated with Br-ORM at 0 and 48 hours and the plates were photographed using a phase-contrast microscope.

### Cell Invasion Assay

Cell invasion assay was performed using BD Biocoat Matrigel Invasion Chambers (BD Biosciences, San Jose, CA, USA), as has been described in the art. Cells were treated with Br-ORM followed by incubation for 24 hours. Cells were fixed using methanol and were stained with crystal violet.

### Western Blot Analysis

The effect of Br-ORM on protein expression in cervical cancer cells were determined by Western blot analysis by using specific antibodies.

### Nuclear and Cytoplasmic Extract Preparation

The nuclear and cytoplasmic lysates were prepared using respective buffers. The cell lysates (40 μg) were subjected to SDS-PAGE and blotted on PVDF membrane. The membranes were further blocked in 5% milk diluted in 1×TBST solution for 1 hour and incubated overnight in primary antibodies. The following day, the membranes were washed and incubated with secondary antibodies for 1 hour, washed again and developed on a UVP system using chemi-luminiscent reagents.

### Immunofluorescence (IF)

Immunofluorescence was performed as has been described in the art. Briefly, cells were fixed and incubated with respective primary antibodies overnight. This was followed by incubation with CY3, or Alexa Fluor 488, donkey secondary antibodies for 1 hour. The images were then captured using a confocal microscope (Nikon Corporation, Melville, NY, USA).

### Molecular Docking

Atomic coordinates for the crystal structure of beta-catenin (PDB ID: 1JDH) were taken from Protein Data Bank (www.rcsb.org) and for Ormeloxifene/Br-Ormeloxifene, 2D and 3D structures were regained from PubChem. Further calculations and file preparations were done according to a previously published protocol. After preparing the coordinate files of both beta-catenin and ligand (Ormeloxifene/Br-Ormeloxifene), it was subjected to docking using AutoDock 4 package. In brief, to deal with different types of interactions that exist between beta-catenin and Br-Ormeloxifene, the Lamarckian genetic algorithm (LGA) was applied. Most favorable free binding energy and orientations of docking lying inside a range of 2.0 Å in root mean square deviation (rmsd) acceptance were used to group the molecule and classified accordingly. Docked complexes were further optimized, validated, and explored using “Receptor-Ligand Interactions” modules present in the script section of Discover Studio 4.0. PyMOL were used for the visualization of molecular interactions that exist in the resultant dock.

### Isolation of RNA and PCR

RNA from cervical cancer cells was isolated using Qiagen kit and quantified using NanoDrop instrument 2000 (Thermo Scientific, Waltham, MA, USA). To analyze the expression of miR-200a in control and Br-ORM treated cells, 100 ng total RNA was reverse transcribed into cDNA using specific primers designed for miRNA analysis (Applied Biosystems, Foster City, CA, USA). The expression of this miRNAs was determined by qRT-PCR using the Taqman PCR master mixture and specific primers designed for detection of mature miRNAs (Applied Biosystems). The expression of miRNA was normalized with the expression of endogenous control, RNU6B.

### Xenograft Study

The anticancer activity of Br-ORM was assessed in an orthotopic mouse model of cervical cancer. 6-week old female athymic nude mice (Jackson laboratory, Bar Harbor, ME, USA) were used. Mice were maintained in a pathogen-free environment and all the procedures were carried out as per the protocol approved by the UTHSC Institutional Animal Care and Use Committee (UTHSC-IACUC). Briefly, a mixture of equal volume of CaSki cells (4×106) and 100 μl matrigel (BD Biosciences) was injected directly into the cervix of each mouse without any surgery. When the tumor volume reached ˜100 mm³, tumor bearing mice were randomly divided into two groups (n=6 per group). Br-ORM (250 μg/mice) and the respective vehicle control (1×PBS) were administered by intra-tumoral injection three times per week. The tumor volume was periodically measured using the ellipsoid volume formula: tumor volume (mm3)=0.5×L×W×H, wherein L is length, W is width, and H is height. Once the tumor volume of control mice reached 1000 mm³, the mice were sacrificed, and their tumors were excised and used for tissue sectioning (5-micron) for histopathology and biochemical analyses.

### Immunohistochemistry (IHC)

The effect of Br-ORM was determined on E-cadherin, β-catenin, Vimentin, Snail, Slug, HPV-E6, HPV-E7, and proliferating cell nuclear antigen PCNA proteins in excised tumors by immunohistochemistry using Biocare kits (Biocare Medical, Concord, CA, USA). Briefly, the tumor tissues were deparaffinized and rehydrated followed by antigen retrieval using a heat induced technique. Samples were incubated overnight for staining with antibodies. The slides were counterstained with hematoxylin, followed by mounting with vecta mount (Vector Laboratories, Burlingame, CA, USA), and visualized.

In Situ hybridization

The expression of miR-200a in FFPE tissues of control and treated xenograft mice was determined by in situ hybridization analysis using Biochain kit (Biochain In Situ hybridization kit) according to the manufacturer's protocol. Briefly, tissues were deparaffinized and fixed in 4% paraformaldehyde and DEPC-PBS for 20 minutes. They were subjected to digestion using 2×SSC and 0.1% triton X for next 25 minutes. The tissues were prehybridized with prehybridized solution provided with the kit for 4 hours at 48° C. This followed the hybridization of the slides with hybridization buffer and digoxigenin labelled probe (Exicon, Woburn, MA, USA) at 45° C. overnight. After stringent washing of tissue slides with various grades of SSC, the slides were blocked using 1×blocking solution provided with the kit. This followed the subsequent incubation of tissues overnight with AP-conjugated anti-digoxigenin antibody. Further, the slides were washed for 5 minutes with 1×alkaline phosphatase buffer twice. The final visualization was carried out with NBT/BCIP (Pierce, Rodkford, IL, USA) followed by nuclear fast red counterstaining. The slides were mounted and analyzed under microscope.

### Statistical Analysis

The data were analyzed by Two-tailed Student t-test and employed to assess the statistical significance between the control and Br-ORM treated groups.

### Results

### Br-ORM Inhibits Proliferation and Clonogenic Potential of Cervical Cancer Cells

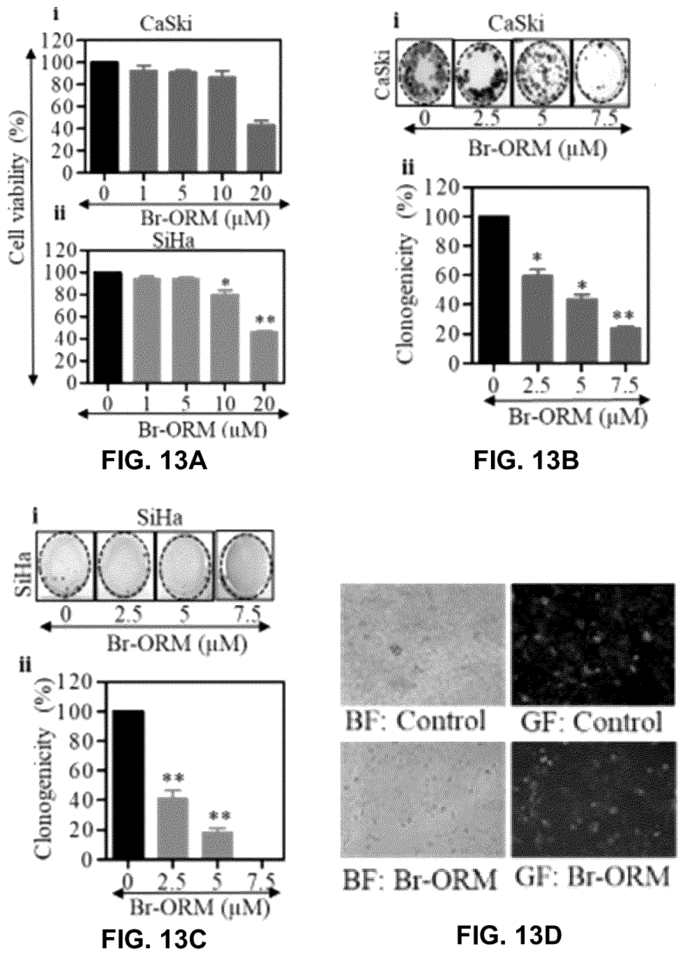

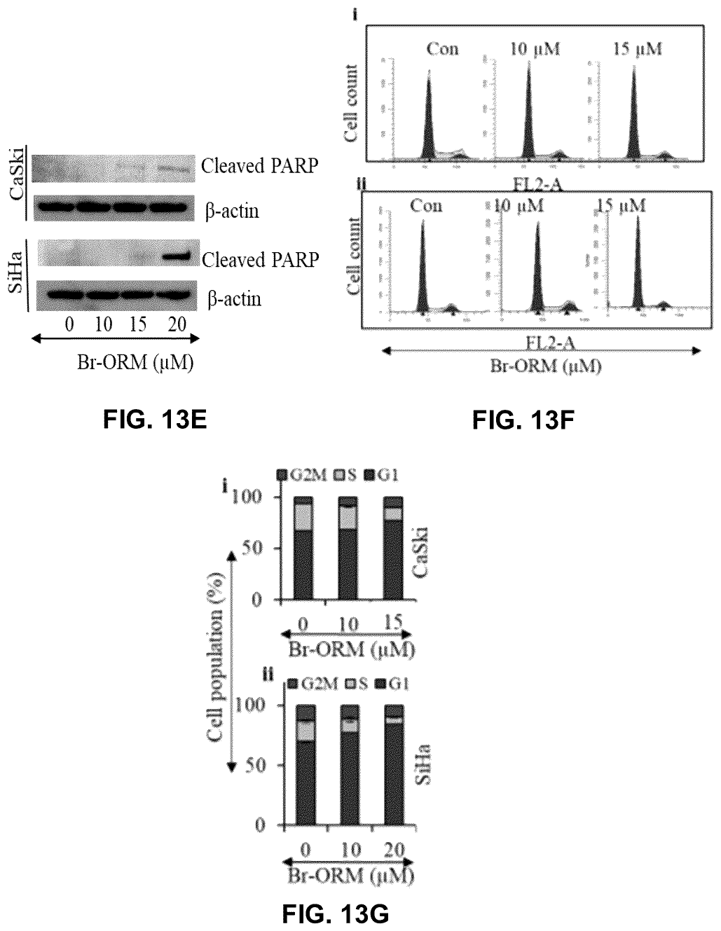

To determine the cytotoxic potential of Br-ORM in cervical cancer cells, MTT assay was employed. As shown in FIG. 13A(i)-(ii), Br-ORM treatment dose dependently inhibited the cell viability of cervical cancer cells. Next, colony formation assay was performed, showing that Br-ORM treatment significantly decreased the number of colonies in both CaSki (FIG. 13B(i)-(ii)) and SiHa cells compared with respective controls (FIG. 13C(i)-(ii)). The effect of Br-ORM on colony formation ability at 5 μM concentration was more significant in SiHa cells compared to CaSki cells. These findings suggest that Br-ORM treatment inhibits the proliferation and clonogeneic potential of cervical cancer cells.

### Br-ORM Induces Apoptosis in Cervical Cancer Cells

It was observed that Br-ORM exerts potent growth inhibitory effects, so the effect of Br-ORM on apoptosis induction was further examined. Cervical cancer cells were treated with Br-ORM (10 μM) for 24 hours and the apoptosis inducing effect of Br-ORM was analyzed by Annexin V staining and Western blot analysis for cleavage in PARP protein. It was revealed that Br-ORM treatment induced apoptosis in CaSki cells as observed by enhanced Annexin V staining (FIG. 13D). Western blot analysis showed that Br-ORM treatment dose dependently enhanced the protein levels of cleaved PARP in CaSki and SiHa cells (FIG. 13E). These results suggest that Br-ORM exhibited apoptosis inducing abilities in cervical cancer cells.

### Br-ORM Arrests Cell Cycle of Cervical Cancer Cells in G1/S Phase

To examine the effect on cell cycle distribution, cervical cancer cells were treated with Br-ORM (10 and 15 μM) and analyzed by flow cytometry (FIG. 13F(i)-(ii)). Results revealed that Br-ORM treatment arrested cell cycle progression of CaSki and SiHa cells at G1/S phase in dose dependent manners in contrast to vehicle control (FIG. 13G(i)-(ii)).

### Br-ORM Inhibits Migratory Potential of Cervical Cancer Cells

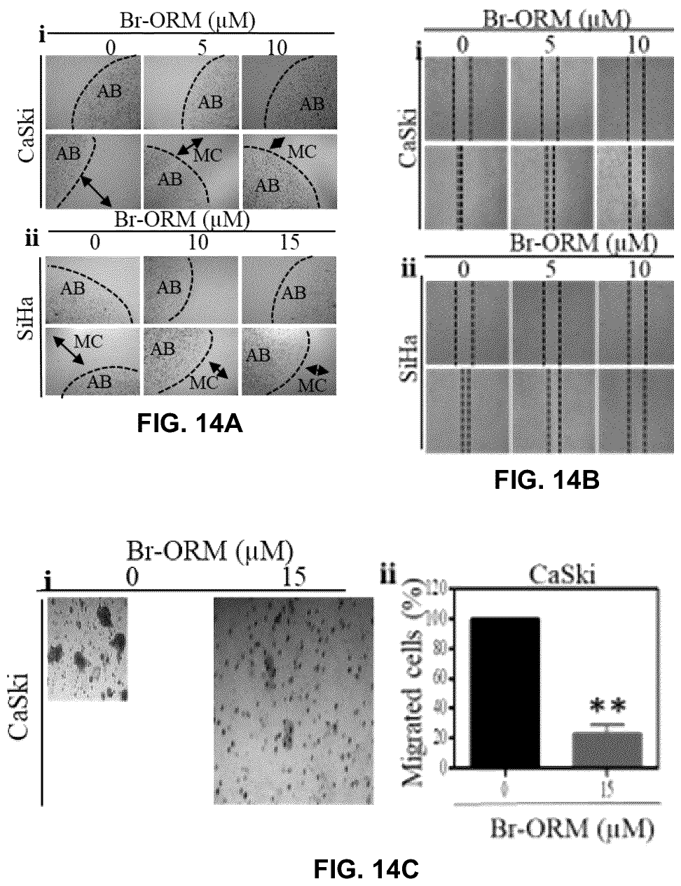

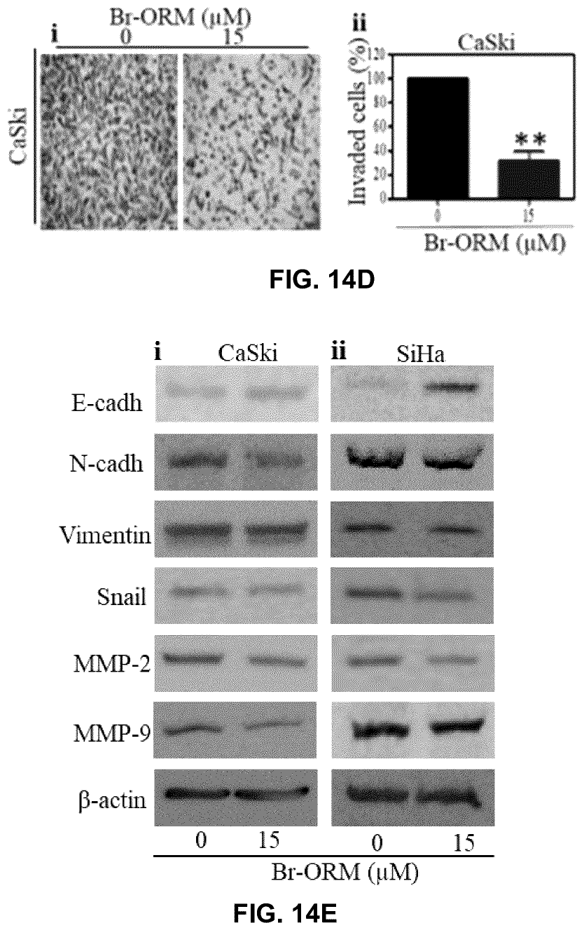

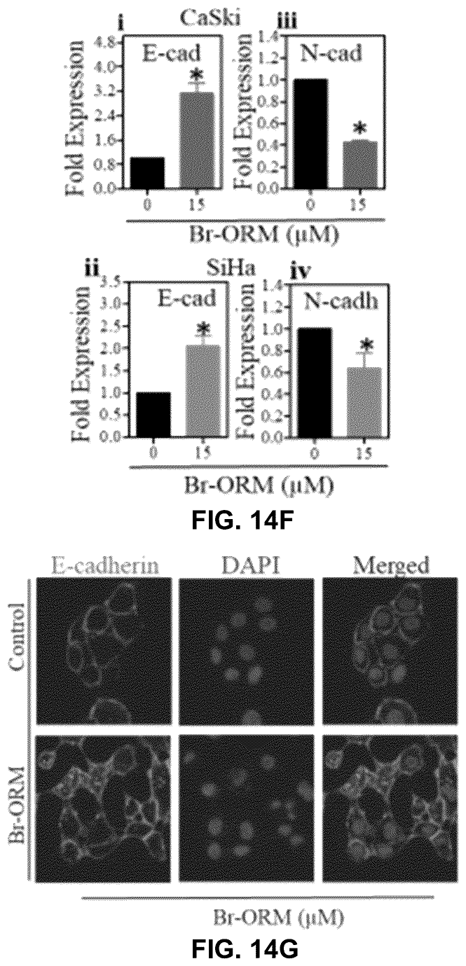

To examine whether Br-ORM treatment inhibits the migratory potential of cervical cancer cells, agarose beads, scratch wound, and Boyden chamber assays were executed. Br-ORM treatment showed dose dependent inhibition of migration abilities in CaSki and SiHa cells. Agarose beads assay showed that Br-ORM effectively inhibited the motility of cervical cancer cells (FIG. 14A(i)-(ii)). Consistently, scratch wound assay experiments showed similar effect at 48 hours, the wound was comparatively more filled in vehicle group than Br-ORM treated group (FIG. 14B (i)-(ii)). Furthermore, the cell migration was also analyzed by Transwell assay coming plate under chemotactic drive which revealed a decrease in migration of cervical cancer cells following the Br-ORM treatment (FIG. 14C(i)-(ii)).

### Br-ORM Inhibits Invasive Potential of Cervical Cancer Cells

Since Br-ORM suppressed the motility of cervical cancer cells, it was investigated whether Br-ORM treatment modulates invasion abilities of cervical cancer cells. Results illustrated that Br-ORM effectively inhibited invasion of cervical cancer cells (FIG. 14D(i)-(ii)). Matrix metolproteinase (MMPs) such as MMP-2 and MMP-9 have been reported to play a critical role in cancer migration and invasion. Activation of Matrix metolproteinase (MMPs) are involved in the extracellular matrix (ECM) degradation that facilitate invasion of cancer cells, thus the effect of Br-ORM treatment on MMPs was investigated and it was found that Br-ORM inhibited the expression of MMP2 and MMP9 (FIG. 14E(i)-(ii)). Taken together, Br-ORM was shown to suppress migration and invasive ability of cervical cancer cells.

### Br-ORM Treatment Effectively Attenuates EMT in Cervical Cancer Cells

Epithelial mesenchymal transition (EMT) is the basic characteristic of cancer cells in which epithelial cells undergo morphologic and molecular changes that transform the cells to mesenchymal, highly metastatic (invasive and motile), and drug-resistant phenotypes. Thus, targeting EMT may reduce the invasive phenotypes of a cancer cell and have significant advantage to overcome drug resistance. It has been reported that β-catenin is involved in invasion and metastasis via inducing EMT in various tumor cells. The effect of Br-ORM treatment on various EMT markers in cervical cancer cells was evaluated. Br-ORM treatment revealed marked inhibition of N-cadherin, vimentin and snail expressions in CaSki (FIG. 14E(i)) and SiHa (FIG. 14E(ii)) whereas it induced the expression of E-cadherin in CaSki (FIG. 14E(i)) and SiHa (FIG. 14E(ii)) as studied by Western blot analysis. The effect of Br-ORM on expression of EMT markers in cervical cancer cells compared to untreated group by use of qPCR analysis was determined. The mRNA level of N-cadherin was significantly reduced (FIG. 14F(iii)-(iv)) and E-cadherin was significantly elevated in both cervical cancer cells (FIG. 14F(i)-(ii)). Expression of E-cadherin in cervical cancer cells treated with Br-ORM (10 μM) for 18 hours was also determined by immunofluorescence and confocal microscopy. Images were captured under 60× magnification and representative images are presented (FIG. 14G). This data showed that Br-ORM inhibits EMT process in cervical cancer by downregulating the levels of N-cadherin, vimentin, and snail, and upregulating E-cadherin.

### Br-ORM Represses β-Catenin Signaling in Cervical Cancer Cells

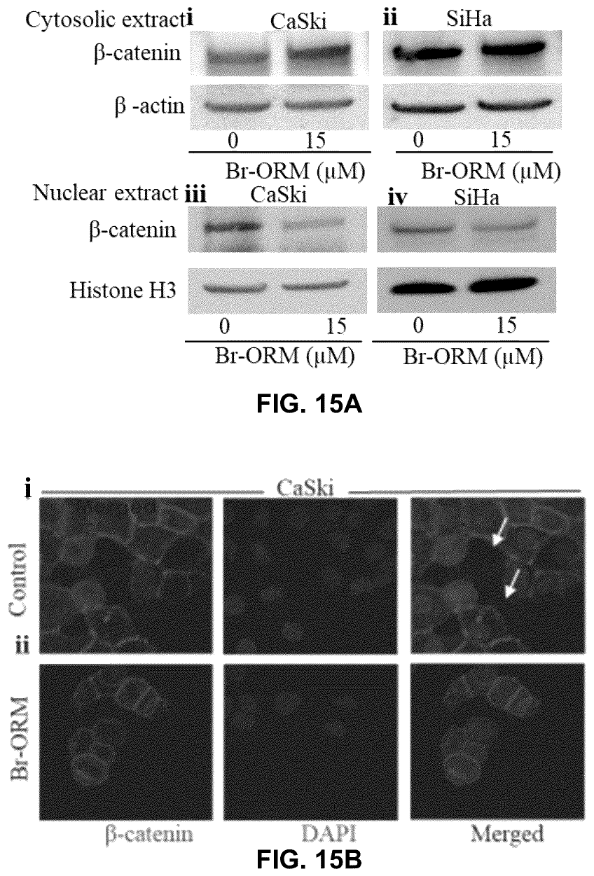

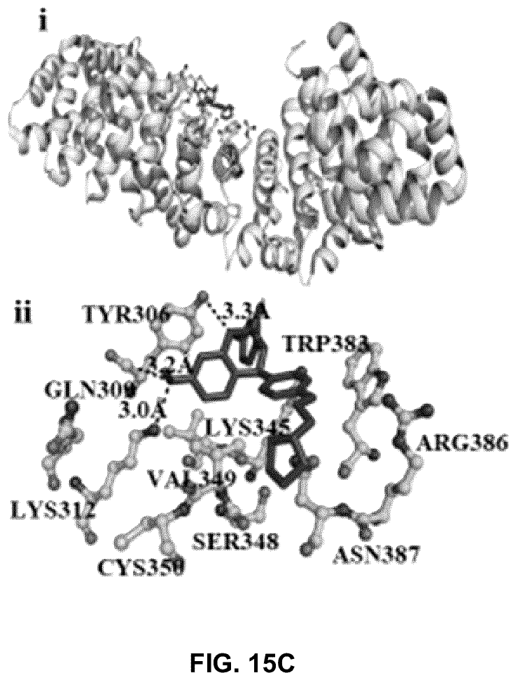

The EMT process in a cell is driven by growth factors or other signaling proteins such as Wnt/beta-catenin and extracellular matrix component that may stimulate cellular growth and migration. The effect of Br-ORM on β-catenin signaling was examined. Br-ORM treatment (15 μM) inhibited nuclear β-catenin in CaSki (FIG. 15A(iii)) and SiHa cells (FIG. 15A(iv)) through its sequestration in the cytoplasm (FIG. 15A(i)-(ii)) as determined by Western blot analysis. This result was further confirmed by confocal microscopy as Br-ORM showed inhibition of β-catenin translocation into the nucleus of cervical cancer cells compared with control (FIG. 15B).

Molecular docking studies were performed to determine the orientation of Br-ORM bound in the active sites of β-catenin. Docking results signified that Br-ORM binds into the binding site region of some known inhibitors of beta-catenin (FIG. 15C(i)-(ii)). The complex formed between the ligand and protein is stabilized by different non-covalent interactions. The binding energy (ΔG) of Br-Ormeloxifene with beta-catenin is 7.6 kcal mol⁻¹. Table 3 indicates that Br-Ormeloxifene binds significantly with beta-catenin. LigPlot analysis of docked structure shows that beta-catenin offers numerous van der Waals interactions to ligand Br-Ormeloxifene. In the binding region, following residues of beta-catenin LYS345, SER348, VAL349, CYS350, TRP383, ARG386 and ASN387 show van der Waals interactions with ligand. Furthermore, TYR306, GLN309 and LYS312 form three hydrogen bonds with Br-Ormeloxifene. These results are shown below in Table 3. The table shows the Br-ORM score with β-catenin as well as the hydrogen bond formation of the binding site residues of the target protein which offer numerous van der Waals interactions with Br-ORM. These bonds provide stability to the protein-ligand complex. Overall, these results suggest that Br-ORM is a potent inhibitor of β-catenin signaling pathway.

TABLE 3

| | | | | Hydrogen | | |
|---|---|---|---|---|---|---|
| | | Binding | No. of | bond | | |
| | | Energy | hydrogen | forming | Distances | Other interacting |
| Protein | Ligand | (kcal/mol) | bonds | residues | (Å) | residues |
| B-catenin | Br-Ormelox- | −7.6 | 3 | TYR306 | 3.3 | LYS345, SER348, |
| (PDB ID: | ifene | | | GLN309 | 3.2 | VAL349, CYS350, |
| 1JDH) | | | | LYS312 | 3.0 | TRP383, ARG386, |
| | | | | | | ASN387 |
| STAT3 | | −7.9 | 1 | ARG325 | 2.8 | ALA250, GLY253, |
| (PDBID: | | | | | | GLU324, ARG325, |
| 1BG1) | | | | | | CYS328, GLN326, |
| | | | | | | PRO330, MET331, |
| | | | | | | ASP333, PRO334, |
| | | | | | | THR346, GLN247, |
| | | | | | | GLN248 |
| HPV16E6 | | −7.5 | 2 | GLN107 | 3.22 | LEU50, LEU67, |
| (PDBID: | | | | SER71 | 3.1 | TYR70, ARG77, |
| 4XR8) | | | | | | HIS78, ARG131 |
| HPV16E7 | | −7.2 | 1 | ASN53 | 2.2 | GLU34, TYR52, |
| (Homology | | | | | | ARG66, GLU80, |
| model) | | | | | | MET84 |

### Br-ORM Downregulates the HPV Oncoproteins in Cervical Cancer Cells

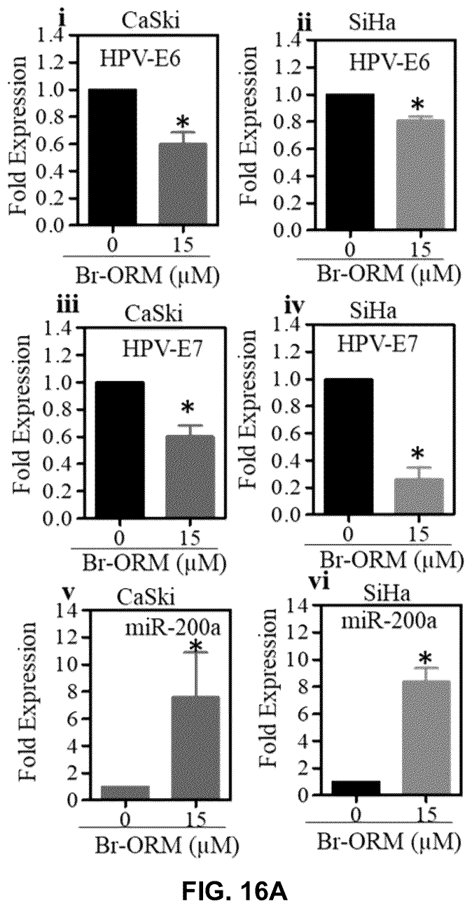

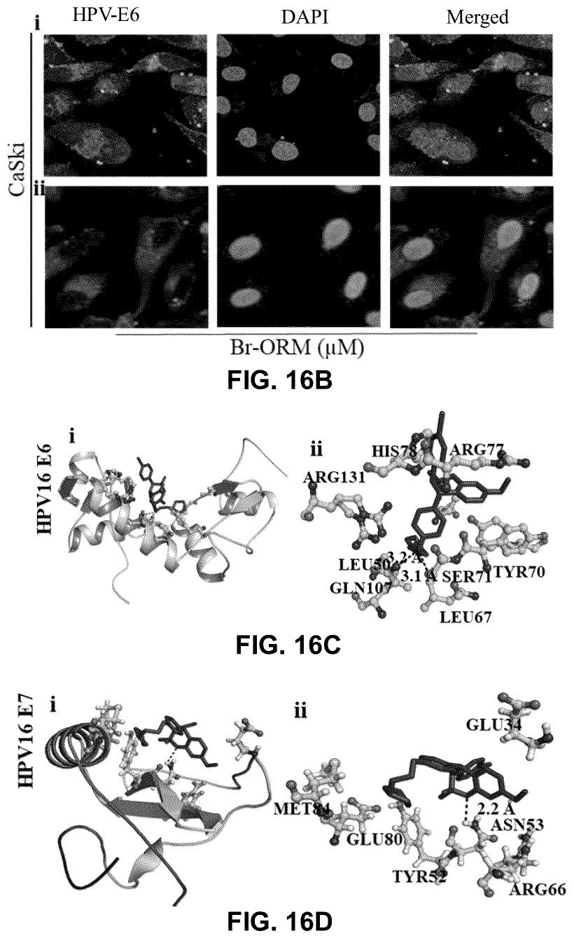

HPV infections are associated with a majority of cervical cancer cases. The effect of Br-ORM on the expression of HPV E6 and HPV E7 oncogenes in CaSki and SiHa cells was investigated. A significant downregulation by Br-ORM of both HPV16 E6 and E7 transcripts via qPCR (FIG. 16A(i)-(iv)) was shown. These results were further confirmed by confocal microscopy (FIG. 161B(i)-(ii)) and molecular docking analysis (FIG. 16C(i)-(ii) and FIG. 16D(i)-(ii)). Br-ORM repressed both transcription and translation of E6 and E7 oncogene in cervical cancer cells.

Br-ORM Treatment Restore Tumor Suppressor miR200a in Cervical Cancer Cells

Modulations of microRNAs are involved in cervical cancer development, progression and metastasis. It is reported that expression of β-catenin is directly regulated by miR-200a. Br-ORM targets β-catenin signaling, therefore, to further understand the underlying mechanisms, the expression of miR-200a following the Br-ORM treatment was evaluated. It was observed that Br-ORM treatment showed a significant (P<0.05) increase in the expression of miR-200a in cervical cancer cells as determined by qPCR analysis (FIG. 16A(v)-(vi)). These results suggest that Br-ORM treatment restored tumor suppressor miR-200a in cervical cancer cells.

### Molecular Docking Studies

Molecular docking studies were performed to see the different type of interactions that existed between amino acid residues of selected target protein and Br-Ormeloxifene. Atomic coordinates for the crystal structure of beta-catenin (PDB ID: 1JDH), HPV16 E6 (PDBID: 4XR8), and transcription factor STAT3 (PDBID: 1BG1) were taken from Protein Data Bank (www.rcsb.org) and for Br-Ormeloxifene, 2D and 3D structures were regained from PubChem (https://pubchem.ncbi.nlm.nih.gov/compound/5281318#section=2D-Structure/3D-Conformer). Further calculations, and file preparations were completed according to a previously published protocol. In the case of HPV16E7, no structure was available, so its three dimensional homology models were predicted using I-TASSER server (2EW1A taken as structure homologue). The homology model was validated using PROCHECK Ramachandran plot and MolProbity Ramachandran analysis. After preparing the coordinate files of all the selected targets and ligand (Br-Ormeloxifene), they were subjected to docking using AutoDock 4 package. Evaluation of docking results helps to assess the intermolecular distance between the interacting residues of protein-ligand complex. Results of docking are shown in Table 3 and these results suggested that Br-Ormeloxifene binds into the binding site cavity of some known inhibitors of beta-catenin, STAT3, HPV1 E6, and HPV1 E7 as reported previously by other groups. The complex formed between the ligand and protein is stabilized by different non-covalent interactions. It was found that Br-Ormeloxifene forms two hydrogen bonds with HPV16E6 and, one with each of HPV16E7 and STAT3 (FIG. 16C(i)-(ii) and FIG. 16D(i)-(ii)). Besides hydrogen bond formation, the binding site residues of these target proteins offer numerous van der Waals interactions to Br-Ormeloxifene (Table 3). Three-dimensional surface representation of Br-Ormeloxifene with these proteins also suggested that it binds to the binding site cavity of each target. The binding energy (AG) of Br-Ormeloxifene with beta-catenin, STAT3, HPV16E6 and HPV16E7 is shown in Table 3, which suggest that Br-Ormeloxifene forms stable complex with these protein targets.

### Br-ORM Inhibits Cervical Cancer Cell Derived Xenograft Tumors in Athymic Nude Mice

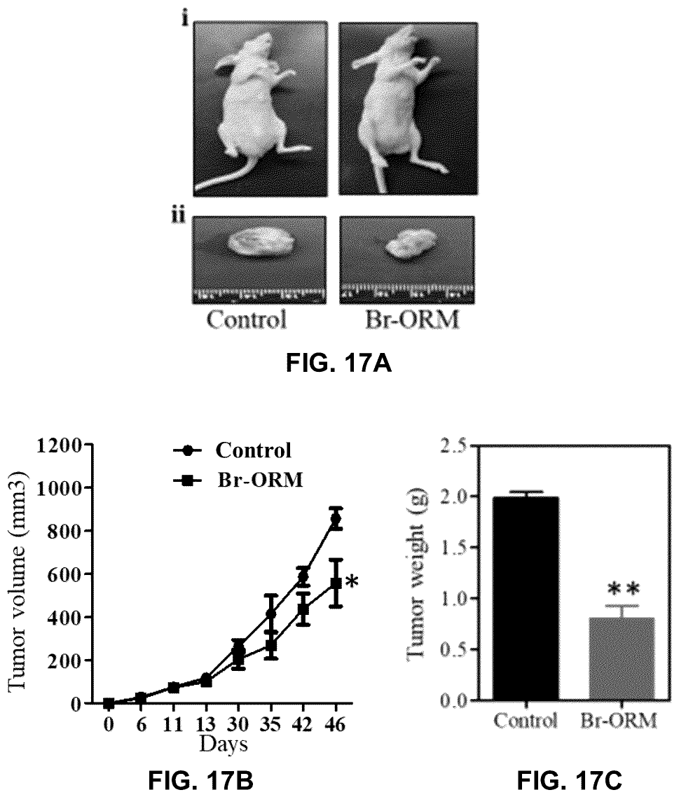

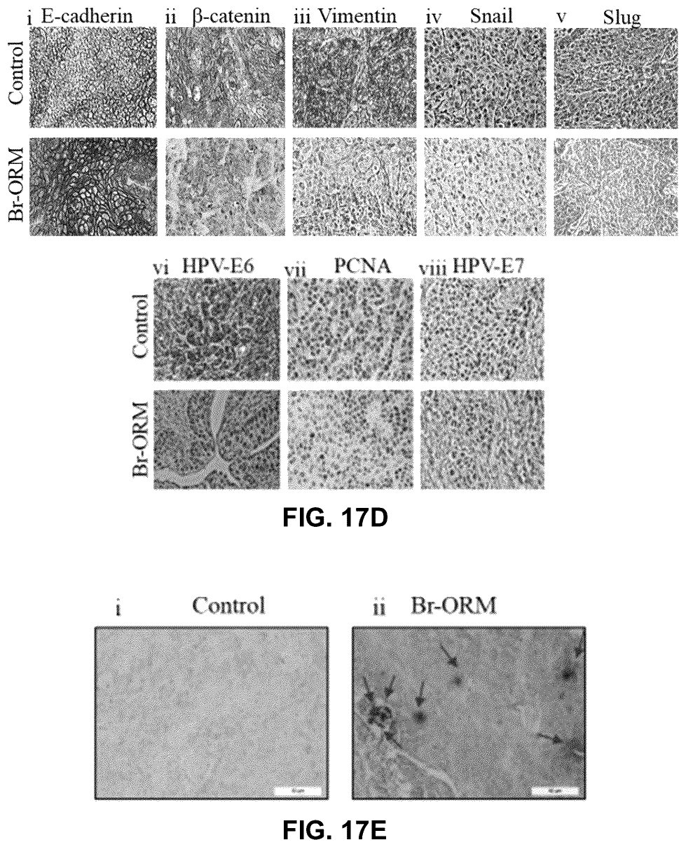

To investigate whether Br-ORM treatment inhibits cervical tumor growth in vivo, we developed an orthotropic xenograft mouse model using CaSki cells as descried above. In this study, mice were treated with Br-ORM (250 μg/mice, three times per week) intratumorally. Intra-tumoral injection of Br-ORM significantly regressed xenograft tumors in athymic nude mice as compared to vehicle treated group (FIG. 17A(i)-(ii)). Br-ORM administration significantly (p<0.05) reduced both tumor volume (FIG. 17B) and tumor weight (FIG. 17C) compared to control groups. Moreover, the effect of Br-ORM on the expression of E-cadherin, β-catenin, vimentin, snail, slug, HPV-E6, HPV-E7, and PCNA were determined. Results showed an increase in the expression of E-cadherin of Br-ORM treated xenograft tumors (FIG. 17D(i)). In addition, there was a significant downregulation in the expression of β-catenin, vimentin, snail, slug, HPV-E6, HPV-E7, and PCNA in Br-ORM treated mice compared to control (FIG. 17D(ii)-(viii)) which is correlated with the in vitro assessment. The tumor tissues were further analyzed for the expression of tumor suppressor miR-200a by in situ hybridization (ISH). Increased expression of miR-200a in Br-ORM treated excised xenograft tumors as compared to control groups (FIG. 17E(i)-(ii)) was found. These results further confirm anti-tumor efficacy of Br-ORM via inhibiting beta-catenin signaling and EMT markers in in vivo model of cervical cancer.

### Discussion

An ORM bromo-analog binds efficiently to the active site of beta-catenin (binding energy −7.6 kcal/mol). Functional studies, discussed above, show that Br-ORM has potential to inhibit the growth and proliferation of HPV-16 positive human cervical cancer (CaSki and SiHa cells). In some aspects, Br-ORM exerts its anti-proliferative and pro-apoptotic impact in a dose-dependent manner. As shown within the examples, Br-ORM treatment inhibited the processes of migration and invasion in cervical cancer cells. In further aspects, Br-ORM treatment arrested cervical cancer cells in G1/S phase of the cell cycle and induced apoptosis. In some embodiments, Br-ORM induces the expression of E-cadherin and inhibits Snail and N-cadherin expression in cervical cancer cells. As further shown within the examples, in some aspects, Br-ORM effectively blocks EMT progression. Further, treatment with Br-ORM has restored miR-200a expression in cervical cancer cells as seen through real-time PCR analysis.

In excised xenograft tumors of Br-ORM treated mice, efficient inhibition of β-catenin and EMT-related markers (vimentin, N-cadherin, snail, and slug) was found. ISH findings showed that therapy with Br-ORM restores miR-200a expression in excised xenograft tumors. Both in vitro and in vivo, Br-ORM replenished the tumor suppressor miR-200a.

The examples further demonstrated that Br-ORM effectively targets signaling pathways related to β-catenin and EMT to repress the development of cervical tumors and metastatic phenotypes. Br-ORM can be used alone or in conjunction with the present therapeutic regimen for the treatment of cervical cancer.

The disclosures being thus described, it will be obvious that the same may be varied in many ways. Such variations are not to be regarded as a departure from the spirit and scope of the disclosures and all such modifications are intended to be included within the scope of the following claims.

### FIGURE LEGENDS

FIG. 1. Organic synthesis of the compounds of the present disclosure.

FIG. 2. Ability of ORM-series to inhibit the growth of A2780 cell lines.

FIGS. 3A, 3B, and FIG. 4. Effect of ORM-Br on β-catenin degradation analyzed by pulse chase experiment.

FIGS. 5A-5D. The effect of Br-ORM on clonogenic potential of cervical cancer cells. Representative colony images of control and Br-ORM treated CaSki (FIG. 5A) and SiHa (FIG. 5B) cells. The bar graphs represent the quantification of colony formation in CaSki (FIG. 5C) and SiHa (FIG. 5D) cells.

FIG. 6. Br-ORM inhibits the growth of cervical cancer (CaSki and SiHa) cells.

FIGS. 7A-7D. Br-ORM inhibits the migration of cervical cancer (CaSki and SiHa) cell. Effect of ORM-Br on motility potential of CaSki and SiHa cells as predicted by agarose bead assay. Images represent the migratory cells (MC) in control and ORM-Br treated groups at 0 and 48 hours. AB means agarose beads. Images magnification were at 4.

FIGS. 8A-8B. Effect of ORM-Br on invasion of CaSki and SiHa cell lines in Boyden chamber and xCELLigence assay. Images represent the invaded cells of control and ORM-Br-treated CaSki and SiHa cells as determined by Boyden Chamber kit. Image magnification was at 20.

FIGS. 9A-9E. Effect of ORM-Br on β-catenin distribution in the cytoplasm and nucleus of CaSki and SiHa cell lines. Cells were subjected to treatment with 15 μM concentration of ORM-Br for 24 hours, nuclear extracts were prepared and western blot analysis were used to detect the protein levels of β-catenin. The results revealed a decrease in expression of nuclear β-catenin and increased expression of β-catenin in the cytoplasm (FIG. 9A and FIG. 9C) of CaSki and SiHa cells. Western blots were reprobed with β-actin and Histone H3 antibodies as an internal control. Effect of ORM-Br on β-catenin localization in CaSki cells as illustrated by confocal microscopy (FIG. 9E).

FIG. 10. Effect of ORM-Br on EMT markers. About 70% of confluent SiHa and CaSki were treated with ORM-Br (10-20 mmol/L) for 24 hours. Cell lysates were prepared, and western blot analysis were conducted for EMT markers and MMPs analysis. Effect of ORM-Br on protein levels of cell-cycle regulatory proteins (cyclin E1 and p27) in both CaSki and SiHa cell lines.

FIG. 11A. ORM and ORM-Br bonding to different amino acids GSK3B binding pocket.

FIG. 11B. ORM-Br forming hydrogen bonding with ASP: 200:B in GSK3B binding pocket.

FIG. 12A. ORM-Br in beta catenin binding pocket.

FIG. 12B. Ormeloxifene and ORM-Br overlaying inside beta catenin binding pocket.

FIG. 13. Br-ORM decreases proliferation and colony formation of cervical cancer cells. FIG. 13A. Effect of Br-ORM on cell viability of CaSki cells (i) and SiHa cells (ii). Briefly, cervical cancer cells were seeded (2500) in each well of 96-well plate and after overnight incubation; cells were treated with indicated concentrations of Br-ORM for 24 hours. Cell viability was assessed by MTS assay. The bar graph represents the percent viable cells compared to the vehicle-treated cells. Each concentration value shown in bar graph is the mean±SE of triplicate wells of each group. FIG. 13B. Effect of Br-ORM on clonogenic potential of cervical cancer cells CaSki (i) and SiHa (ii). In brief, 500 cells were seeded in each well of 6-well plates. After 3 days, cells were treated with indicated concentrations of Br-ORM and colonies obtained were stained with hematoxylin. Photographs were taken by UVP-gel documentation system for all cells. FIG. 13C. Bar graphs indicating quantification of colony formation in CaSki (i), and SiHa (ii) cells of cervical cancer with respect to vehicle control. FIG. 13D. Effect of Br-ORM on apoptosis in cervical cancer cells. Briefly, cervical cancer cells were treated with Br-ORM (10 μM) for 24 hours and the apoptosis inducing effect of Br-ORM was analyzed by Annexin V staining. FIG. 13E. Cervical cancer cells were treated with the indicated concentrations of Br-ORM for 24 hours; total cell lysates were prepared and subjected to Western blot analysis of apoptosis regulatory protein cleavage of PARP in both CaSki (i) and SiHa (ii). The equal loading of protein in each lane was determined by probing the blots with β-actin antibody (FIG. 13F). Effect of Br-ORM on cell cycle progression in cervical cancer cells. Briefly, Cervical cancer cells were treated with indicated concentration of Br-ORM for 24 hours. Br-ORM arrests cell cycle in G1/S phase as determined by flow cytometry. Histogram and bar graph represent cell-cycle distribution in CaSki (FIG. 13G(i)), and SiHa (FIG. 13G(ii)) cells of cervical cancer. Experiments were performed in triplicate.

FIG. 14. Br-ORM decreases migration, invasion and attenuates EMT in cervical cancer cells. Effect of Br-ORM on cell migration of cervical cancer cells as determined by agarose bead (FIG. 14A), scratch wound healing (FIG. 14B), and Boyden chamber assay (FIG. 14C). AB denotes agarose bead while MC denotes migratory cells. Arrows indicate the distance of cervical cancer (CaSki and SiHa) cells migration in control and Br-ORM treated groups (FIG. 14A(i)-(ii)). Scratch wound assay showing effect of Br-ORM on CaSki and SiHa cells. Briefly, a standardized wound was made using a 200 μl micropipette tip in 80-90% confluent 12-well plate and treated with indicated concentration of Br-ORM. Closure of wound was determined and photographed using phase contrast microscopy. Representative images of migratory CaSki and SiHa cells in control and Br-ORM treated groups at 0, 24 and 48 hours (FIG. 14B(i)-(ii)). Representative images of migratory cells in control and Br-ORM treated groups at 0 and 48 hours (FIG. 14C(i)). Bar graph represent the quantification of migrated CaSki cells of control and Br-ORM treated groups (FIG. 14C(ii)). FIG. 14D. Effect of Br-ORM treatment on invasion of CaSki cells as determined by a commercially available kit (BD Biosciences) as described in materials and methods. In brief, 24 hours post-treatment of Br-ORM, invaded cells were fixed, stained and counted. Representative images of invaded control and Br-ORM treated cells (FIG. 14D(i)). Bar graph represent the quantification of invaded CaSki cells of control and Br-ORM treated groups (FIG. 14D(ii)). FIG. 14E. Effect of Br-ORM treatment on various EMT markers and MMPs in cervical cancer cells. Briefly, 70% confluent cervical cancer cells were treated with Br-ORM for 24 hours. Cell lysates were prepared and subjected to Western blot analysis for EMT markers and MMPs in CaSki and SiHa cells (FIG. 14E(i)-(ii)). FIG. 14F. Cervical cancer cells were treated with Br-ORM for 18 hours and then RNA was isolated and subjected to qPCR for E-cadherin (14F(i)-(ii)), and N-cadherin (FIG. 14F(iii)-(iv)). FIG. 14G. Effect of Br-ORM on E-cadherin expression in cervical cancer cells as determined by confocal microscopy in control and Br-ORM treated cells after 18 hours.

FIG. 15. Effect OF Br-ORM on β-catenin signaling and molecular docking of Br-ORM with β-catenin. Effect of Br-ORM on β-catenin distribution in cytoplasm and nucleus of CaSki and SiHa cells. Briefly, cervical cancer cells were treated with indicated concentration of Br-ORM for 24 hours and the cell lysates (40 μg) were prepared and subjected to Western blot analysis to detect the protein level of β-catenin. Results indicating Br-ORM treatment (15 μM) inhibited nuclear β-catenin expression in CaSki (FIG. 15A(iii)) and SiHa cells (FIG. 15A(iv)) through its sequestration in the cytoplasm (FIG. 15A(i)-(ii)). Blots were re-probed with Histone H3 and 0-actin antibodies as an internal control. FIG. 15B. Effect of Br-ORM on β-catenin localization in CaSki cells as determined by confocal microscopy (FIG. 15B(i)-(ii)). White arrows indicate localization of β-catenin in control and Br-ORM treated cells after 18 hours (FIG. 15B). Molecular docking study with β-catenin. Cartoon representation of Br-ORM in-complexed with β-catenin (FIG. 15C(i)). Three-dimensional view of different interacting residues of β-catenin with Br-Ormeloxifene. Compound Br-ORM is shown in stick model, residues participating in polar contacts and other interactions are shown in ball and stick, respectively (FIG. 15C(ii)).

FIG. 16. Effect of Br-ORM on HPV oncoproteins and tumor suppressor miR-200 in cervical cancer cells. Briefly, cervical cancer cells were treated with Br-ORM at indicated concentration for 18 hours and subjected for qPCR analysis to check the mRNA expression levels of HPV E6 and HPV E7 in CaSki and SiHa cells (FIG. 16A(i)-(iv)). GAPDH RNA was used for normalization. Data presented as mean±SEM and are representative of three independent experiments. Increase expression of tumor suppressor miR200a in cervical cancer cells treated with BR-ORM for 24 hours in comparison to non-treated controls was determined through real time PCR (FIG. 16A(v)-(vii)). Data presented as ±SEM are representative of three independent experiments. FIG. 16B. Inhibition of nuclear translocation of HPV E6 by Br-ORM treatment. For this study, 80,000 cervical cancer cells were seeded overnight in 4-well chamber slides, then treated with Br-ORM for 18 hours. Subsequently, they were fixed and processed for immunostaining using anti HPV E6 antibody. Confocal images are shown for nuclear staining showing DAPI and HPV E6, and the merged images, using a Nikon confocal microscope (FIG. 16B(i)-(ii)). FIG. 16C-16D. Molecular docking study with HPV16 E6 and E7 protein. Cartoon representation of Br-ORM in-complexed with E6 (FIG. 16C(i)). Three-dimensional view of different interacting residues of E6 with Br-ORM. Compound Br-ORM is shown in stick model, residues participating in polar contacts and other interactions are shown in ball and stick, respectively (FIG. 16C(ii)). Docking analysis with HPV16 E7 protein. Schematic diagram of Br-ORM docking with HPV16 E7 showing residues involved in hydrogen-bonding, Van der Waals interactions and charge or polar interactions which are represented by various shading (FIG. 16D(i)-(ii)).

FIG. 17 Br-ORM inhibits cervical tumor growth in xenograft mouse model. FIG. 17A. Effect of Br-ORM on CaSki cells derived xenograft tumors in female athymic nude mice. In brief, 12 mice were used in this experiment and were divided into 2 groups: A total of 4×10⁶ CaSki cells were injected directly into the cervix of each mouse without any surgery. Br-ORM was administered (250 μg intra-tumorally three times per week) and control group mice received 0.2 ml PBS. Representative mouse picture of control and Br-ORM treated tumor bearing mouse (FIG. 17A(i)-(ii)). FIG. 17B Line graph indicates regression of CaSki cell-derived xenograft tumor volume in Br-ORM treated mice compared to control group. Values in bar graph represent mean±SE of six mice tumors in each group. FIG. 17C Bar graph representing tumor weight of control and Br-ORM treated mice. FIG. 17D. Effect of Br-ORM on the expression of, E-cadherin, β-catenin, Vimentin, Snail, Slug, PCNA, HPV E6, and HPV E7 were further analyzed immunohistochemistry (IHC) in excised tumors of control and Br-ORM treated mice (FIG. 17D(i)-(viii)). FIG. 17E. Effect of Br-ORM on the expression of miR-200a in control and treated mice excised tumors as determined by in situ hybridization. Student's t-test was performed to analyze significant difference.

What is claimed is:

1. An anticancer composition comprising a compound having the structure: 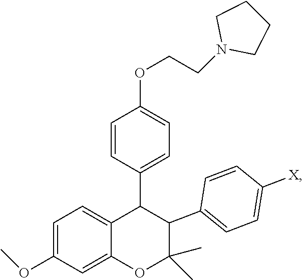
wherein x is an electron withdrawing group;
or a salt, solvate, enantiomer, diastereoisomer, geometric isomer, or tautomer thereof.

2. The composition of claim 1, wherein the electron withdrawing group is selected from F, Cl, Br, and I.

3. The composition of claim 2, wherein the electron withdrawing group is Br.

4. The composition of any one of claims 1-3, further comprising a pharmaceutically acceptable carrier.

5. The composition of any one of claims 1-4, wherein the compound has greater binding affinity for at least one of EGFR, GSK3B, CDK2, STAT3 and/or mTOR, relative to the binding affinity of Ormeloxifene.

6. The composition of any one of claims 1-5, wherein the composition is a Selective Estrogen Receptor Modulator (SERM).

7. The composition of claim 6, further comprising one or more anti-cancer agents.

8. A Selective Estrogen Receptor Modulator (SERM) comprising a compound having the structure: 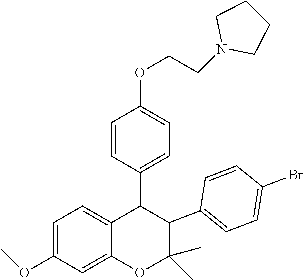 [SMILES: COc1ccc2c(c1)OC(C)(C)C(c1ccc(Br)cc1)C2c1ccc(OCCN2CCCC2)cc1]
and a pharmaceutically acceptable carrier thereof.

9. The composition of claim 8, wherein SERM binds to at least one of EGFR, GSK3B, CDK2, STAT3 and/or mTOR, with a binding affinity greater than that of Ormeloxifene.

10. A method for treating or preventing cancer in a subject in need thereof, the method comprising administering to the subject an effective amount of a compound having the structure: 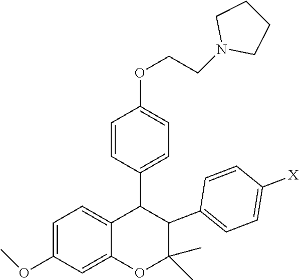
wherein x is an electron withdrawing group;
or a salt, solvate, enantiomer, diastereoisomer, geometric isomer or tautomer thereof.

11. The method of claim 10, wherein the electron withdrawing group is selected from F, Cl, Br, and I.

12. The method of claim 11, wherein the electron withdrawing group is Br.

13. The method of any one of claims 10-12, wherein the cancer is cervical cancer or ovarian cancer.

14. The method of any one of claims 10-12, wherein the cancer is selected from melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-Hodgkin's lymphoma, hepatocellular carcinoma, bladder cancer, testicular cancer, oropharyngeal cancer, head and neck cancer, and lung cancer.

15. The method of any one of claims 10-14, further comprising administering the composition in conjunction with at least one other treatment or therapy.

16. The method of claim 15, wherein the other treatment or therapy comprises co-administering at least one second anti-cancer agent.

17. The method of claim 16, wherein the co-administration of the composition and the at least one second anti-cancer agent provides a synergistic effect.

18. The method of any one of claims 10-17, wherein the administration of the composition inhibits cancer cell migration in the subject.

19. The method of any one of claims 10-18, wherein administration of the composition inhibits cancer cell invasion in the subject.

20. The method of any one of claims 10-19, wherein the composition is administered by oral administration, intravenous administration, intradermal injection, intramuscular injection or subcutaneous injection.
